(19) United States
(12) Patent Application Publication
(10) Pub. No.: US 2016/0045570 A1
(43) Pub. Date: Feb. 18, 2016

(54) BIOMARKERS PREDICTIVE OF THERAPEUTIC RESPONSIVENESS TO IFNB AND USES THEREOF

(71) Applicant: Biogen MA Inc., Cambridge, MA (US)

(72) Inventors: Steven Bushnell, Medfield, MA (US); Alexander Michael Buko, Worcester, MA (US); Eric Taylor Whalley, Charlestown, MA (US); Christopher Stebbins, Newton, MA (US); Zhenming Zhao, Irving, TX (US); Diego Cadavid, Concord, MA (US)

(21) Appl. No.: 14/798,993

(22) Filed: Jul. 14, 2015

Related U.S. Application Data
(63) Continuation of application No. 13/441,745, filed on Apr. 6, 2012, now abandoned.
(60) Provisional application No. 61/474,242, filed on Apr. 11, 2011.
(60) Provisional application No. 61/473,723, filed on Apr. 8, 2011.

(51) Int. Cl. A61K 38/21; A61K 45/06; G01N 33/564

(52) U.S. Cl. CPC A61K 38/215; G01N 33/564; A61K 45/06; G01N 2800/50; G01N 2800/52; G01N 2800/285; G01N 2333/523; G01N 2333/70575

(57) ABSTRACT

Methods, assays and kits for the identification, assessment and/or treatment of a subject having multiple sclerosis (MS) (e.g., a patient with relapsing-remitting multiple sclerosis (RRMS)) are disclosed.

### CROSS-REFERENCE TO RELATED APPLICATIONS

This application is a Continuation of U.S. application Ser. No. 13/441,745, filed Apr. 6, 2012, which claims the benefit of priority to U.S. Provisional Application Ser. No. 61/473,723, filed on Apr. 8, 2011, and U.S. Patent Application Ser. No. 61/474,242, filed on Apr. 11, 2011, both of which are entitled “Biomarkers Predictive of Therapeutic Responsiveness to IFNβ and Uses Thereof.” The contents of the aforesaid applications are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Apr. 4, 2012, is named B2047710.txt and is 2,118 bytes in size.

### BACKGROUND OF THE INVENTION

Multiple sclerosis (MS) is an inflammatory disease of the brain and spinal cord characterized by recurrent foci of inflammation that lead to destruction of the myelin sheath. In many areas, nerve fibers are also damaged. Inflammatory activity in MS patients tends to be highest in the initial phase of disease.

Emerging data demonstrate that irreversible axonal loss occurs early in the course of MS. Transected axons fail to regenerate in the central nervous system (CNS). Therefore, early treatment aimed at suppressing MS lesion formation is of significant importance. As early as disease onset, axons are transected in lesions with active inflammation (Trapp et al., (1998) N Engl J Med 338: 278-285; Bjartmar et al., (2001) Curr Opin Neurol 14: 271-278; Ferguson et al., (1997) Brain 120: 393-399). The degree of demyelination is related to the degree of inflammation and the exposure of demyelinated axons to the inflammatory environment, as well as non-inflammatory mediators (Trapp et al., (1998) N Engl J Med 338: 278-285; Kornek et al., (2000) Am J Pathol 157: 267-276; Bitsch et al., (2000) Brain 123: 1174-1183). There is also destruction of oligodendrocytes with impaired remyelination in demyelinating lesions (Peterson et al., (2002) J Neuropathol Exp Neurol 61: 539-546; Chang et al., (2002) N Engl J Med 346: 165-173). The loss of oligodendrocytes leads to a reduction in the capacity to re-myelinate and may result in the loss of trophic factors that support neurons and axons (Bjartmar et al., (1999) J Neurocytol 28: 383-395).

Given the destructive effects of inflammatory MS lesions, the need exists for identifying and/or assessing a patient or patient population having multiple sclerosis that would benefit from treatment with an interferon-β (IFN-(3) agent in the course of disease, or identifying a patient or patient population as responding or not responding to an IFN-13 agent.

### SUMMARY OF THE INVENTION

The present invention provides, at least in part, methods, assays and kits for the identification, assessment and/or treatment of a subject having multiple sclerosis (MS) (e.g., a subject with relapsing-remitting multiple sclerosis (RRMS)). In one embodiment, responsiveness of a subject to an interferon beta agent (referred to interchangeably herein as an “IFN-β,” “IFN-b,” “IFNβ,” or “IFNb,” agent), e.g., an IFN-β 1a molecule or an IFN-β 1b molecule, is predicted by evaluating an alteration (e.g., an increased or decreased level) of an MS biomarker in a sample, e.g., a serum sample obtained from an MS patient. In certain embodiments, the MS biomarker evaluated is Chemokine (C-C motif) ligand 21 (CCL21) and/or B Cell (Lymphocyte) Activating Factor) (BAFF), and (optionally) one or more of: Interleukin-1 Receptor Antagonist (IL-1RA), Interleukin-13 (IL-13), Monocyte Chemoattractant Protein-1 (MCP-1), C-reactive protein (CRP), Beta-2-microglobulin (B2M), ferritin, and/or Tumor necrosis factor receptor-2 (TNFR2). Thus, the invention can, therefore, be used, for example: To evaluate responsiveness to, or monitor, a therapy or treatment that includes an IFN-b agent; identify a patient as likely to benefit from a therapy or treatment that includes an IFN-b agent; stratify patient populations (e.g., stratify patients as being likely or unlikely to respond (e.g., responders vs. non-responders) to a therapy or treatment that includes an IFN-b agent; and/or more effectively monitor, treat multiple sclerosis, or prevent worsening of disease and/or relapse.

Accordingly, in one aspect, the invention features a method of, or assay for, evaluating a sample, e.g., a sample from an MS patient. The method includes detecting an alteration (e.g., an increased or decreased level) of an MS biomarker in the sample. In one embodiment, the MS biomarker evaluated includes CCL21 and/or BAFF, and optionally, one or more of: IL-1RA, IL-13, MCP-1, CRP, B2M, ferritin, and/or TNFR2.

The method, or assay, can further include one or more of the following:

(i) identifying a subject (e.g., a patient, a patient group or population), having MS, or at risk of developing MS, as having an increased or a decreased likelihood to respond to an MS treatment (or an MS therapy, as used interchangeably herein), e.g., identifying a subject as a responder or a non-responder to the MS treatment;

(ii) determining a treatment regimen upon evaluation of the sample (e.g., selecting, or altering the course of, a therapy or treatment, a dose, a treatment schedule or time course, and/or the use of an alternative MS therapy);

(iii) analyzing a time course of MS disease progression in the subject; and/or

(iv) treating the subject (e.g., administering an MS therapy to the subject).

In one embodiment, the MS treatment includes a treatment with an IFN-b agent.

In one embodiment, one or more of (i)-(iv) are determined in response to the detection of the alteration. An alteration (e.g., an increased or a decreased level) in the sample in one or more of the aforesaid MS biomarkers relative to a specified parameter (e.g., a reference value or sample; a sample obtained from a healthy subject; or a sample obtained from the subject at a different time interval, e.g., prior to, during, or after treatment), indicates one or more of: an increased or decreased responsiveness of the subject to the IFN-b agent; identifies the subject as having an increased or decreased likelihood to respond to the treatment with the IFN-b agent; determines the treatment to be used; and/or analyzes or predicts the time course of the MS disease.

In another aspect, the invention features a method of, or assay for, identifying a subject (e.g., a patient, a patient group or population), having MS, or at risk for developing MS, as having an increased or decreased likelihood to respond to an MS treatment, e.g., an MS treatment with an IFN-b agent. The method includes:

acquiring a value (e.g., obtaining possession of, determining, detecting, or evaluating, the level) of an MS biomarker in a subject (e.g., a sample from the subject), and

responsive to said value, identifying the subject having MS, or at risk for developing MS, as being likely or less likely to respond to an IFN-b agent.

In one embodiment, the MS biomarker evaluated includes CCL21 and/or BAFF, and optionally, one or more of: IL-1RA, IL-13, MCP-1, CRP, B2M, ferritin, and/or TNFR2. An increased or a decreased value in one or more of the aforesaid MS biomarkers relative to a specified parameter (e.g., a reference value or sample; a sample obtained from a healthy subject; or a sample obtained from the subject at a different time interval, e.g., prior to, during, or after treatment), indicates an increased or decreased responsiveness of the subject to the IFN-b agent.

In another aspect, the invention features a method of, or assay for, evaluating or monitoring a treatment (e.g., an MS treatment, e.g., an MS treatment with an IFN-b agent) in a subject (e.g., a patient, a patient group or population), having MS, or at risk for developing MS. The method includes:
acquiring a value (e.g., obtaining possession of, determining, detecting, or evaluating, the level) of an MS biomarker in a subject (e.g., a sample from the subject); and

(optionally) responsive to said value, treating, selecting and/or altering one or more of the course of the MS treatment, the dosing of the MS treatment, the schedule or time course of the MS treatment, or administration of a second, alternative MS therapy.

In one embodiment, the MS biomarker evaluated includes CCL21 and/or BAFF, and optionally, one or more of: IL-1RA, IL-13, MCP-1, CRP, B2M, ferritin, and/or TNFR2. In one embodiment, the method includes comparing the value of the MS biomarker to a specified parameter (e.g., a reference value or sample; a sample obtained from a healthy subject; or a sample obtained from the subject at a different time interval, e.g., prior to, during, or after treatment). The method can be used, e.g., to evaluate the suitability of, or to choose between alternative treatments, e.g., a particular dosage, mode of delivery, time of delivery, or generally to determine the subject's probable drug response.

In yet another aspect, the invention features a method of, or assay for, evaluating a subject's prognosis or MS disease progression, in a subject (e.g., a patient, a patient group or population), having MS, or at risk for developing MS. The method includes:

acquiring a value (e.g., obtaining possession of, determining, detecting, or evaluating, the level) of an MS biomarker in a subject (e.g., a sample from the subject); and

(optionally) comparing the value of the MS biomarker to a specified parameter (e.g., a reference value or sample; a sample obtained from a healthy subject; or a sample obtained from the subject at different time intervals, e.g., prior to, during, or after treatment, e.g., an MS treatment, e.g., an MS treatment with an IFN-b agent).

In certain embodiments, the sample is obtained at different time intervals, e.g., prior to, during, or after treatment with an MS therapy. In one embodiment, the MS biomarker evaluated includes CCL21 and/or BAFF, and optionally, one or more of: IL-1RA, IL-13, MCP-1, CRP, B2M, ferritin, and/or TNFR2. An increased or a decreased value in one or more of the aforesaid MS biomarkers relative to a specified parameter (e.g., a reference value or sample; a sample obtained from a healthy subject; or a sample obtained from the subject at different time intervals, e.g., prior to, during, or after treatment), indicates an increased or decreased disease progression in the subject in response to the MS therapy, e.g., a therapy with an IFN-b agent.

### Treatment

In other embodiments, any of the aforesaid methods further include treating, or preventing in, a subject having MS one or more symptoms associated with MS. In certain embodiments, the treatment includes reducing, retarding or preventing, a relapse, or the worsening of a disability, in the MS subject. In one embodiment, the method includes, responsive to an MS biomarker value (e.g., an MS biomarker value obtained as described herein), administering to the subject (e.g., a patient with relapsing-remitting multiple sclerosis (RRMS)) a therapy for MS (also referred to herein as an “MS therapy”), e.g., an MS therapy with an IFN-b agent, in an amount sufficient to reduce one or more symptoms associated with MS.

In yet another aspect, the invention features a method of treating or preventing one or more symptoms associated with MS, in a subject having MS, or at risk for developing MS. The method includes:

acquiring a value (e.g., obtaining possession of, determining, detecting, or evaluating the level) of an MS biomarker chosen from CCL21 and/or BAFF, and optionally, one or more of: IL-1RA, IL-13, MCP-1, CRP, B2M, ferritin, and/or TNFR2, in a subject;

responsive to said value, administering to a subject (e.g., a patient with relapsing-remitting multiple sclerosis (RRMS)) a therapy for MS (also referred to herein as an “MS therapy”), e.g., an MS therapy with an IFN-b agent, in an amount sufficient to reduce one or more symptoms associated with MS.

In certain embodiments, the method of treatment includes an MS therapy, e.g., an MS therapy that includes an IFNβ agent (e.g., an IFN-β 1a molecule or an IFN-β 1b molecule, including analogues and derivatives thereof (e.g., pegylated variants thereof)). In one embodiment, the MS therapy includes an IFN-β 1a agent (e.g., AVONEX®, REBIF®). In another embodiment, the MS therapy includes an INF-β 1b agent (e.g., BETASERON®, BETAFERON®). In another embodiment, the MS therapy is an alternative therapy (e.g., a therapy selected when a patient is non-responsive to an INF-β therapy).

In one embodiment, the MS therapy is a disease modifying MS therapy. In certain embodiments, the MS therapy is an alternative therapy to the IFN-β agent. In one embodiment, the alternative therapy includes a polymer of four amino acids found in myelin basic protein, e.g., a polymer of glutamic acid, lysine, alanine and tyrosine (e.g., glatiramer (COPAXONE®)). In other embodiments, the alternative therapy includes an antibody or fragment thereof against alpha-4 integrin (e.g., natalizumab (TYSABRI®). In yet other embodiments, the alternative therapy includes an anthracenedione molecule (e.g., mitoxantrone (NOVANTRONE®)). In yet another embodiment, the alternative therapy includes a fingolimod (e.g., FTY720; GILENYA®). In one embodiment, the alternative therapy is a dimethyl fumarate (e.g., an oral dimethyl fumarate (BG-12)). In other embodiments, the alternative therapy is an antibody to the alpha subunit of the IL-2 receptor of T cells (e.g., Daclizumab). In yet other embodiments, the alternative therapy is an antibody against CD52 (e.g., alemtuzumab (LEMTRADA®)). In yet another embodiment, the alternative therapy includes an anti-LINGO-1 antibody.

In certain embodiments, the method further includes the use of one or more symptom management therapies, such as antidepressants, analgesics, anti-tremor agents, among others.

Additional embodiments or features are as follows:

In certain embodiments, the MS biomarker evaluated, using the methods or assays disclosed herein includes, or consists of, CCL21. In other embodiments, the MS biomarker evaluated, using the methods or assays includes, or consists of, BAFF. In other embodiments, the MS biomarker evaluated includes, or consists of CCL21 and BAFF. In yet other embodiments, the MS biomarker evaluated includes CCL21 or BAFF, and one, two, three, four, five, six, seven or all of: IL-1RA, IL-13, MCP-1, CRP, B2M, ferritin, or TNFR2. In yet other embodiments, the MS biomarker evaluated includes CCL21 and BAFF, and one, two, three, four, five, six or all seven of: IL-1RA, IL-13, MCP-1, CRP, B2M, ferritin, or TNFR2.

The method or assays disclosed herein can further include one or more steps of: performing a neurological examination, evaluating the subject's status on the Expanded Disability Status Scale (EDSS), or detecting the subject's lesion status (e.g., as assessed using an MRI).

For any of the methods or assays disclosed herein, the subject treated, or the subject from which the sample is obtained, is a subject having, or at risk of having MS at any stage of treatment. In certain embodiments, the MS patient is chosen from a patient having one or more of: Benign MS, RRMS (e.g., quiescent RRMS, active RRMS), primary progressive MS, or secondary progressive MS. In other embodiments, the subject has MS-like symptoms, such as those having clinically isolated syndrome (CIS) or clinically defined MS (CDMS). In one embodiment, the subject is an MS patient (e.g., a patient with RRMS) prior to administration of an MS therapy described herein (e.g., prior to administration of an IFN-b agent). In one embodiment, the subject is a newly diagnosed RRMS patient, e.g., a newly diagnosed RRMS patient prior to IFN-b therapy. In another embodiment, the subject is an MS patient (e.g., an RRMS patient) after administration of an MS therapy described herein (e.g., IFN-b agent). In other embodiments, the subject is an MS patient after administration of the MS therapy for one, two weeks, one month, two months, three months, four months, six months, one year or more.

The methods, or assays, described herein can be used to distinguish MS from other neurological conditions, e.g., to distinguish MS from CIS.

In certain embodiments, the method, or assay, further includes the step of obtaining the sample, e.g., a biological sample, from the subject. In one embodiment, the method, or assay, includes the step of obtaining a predominantly non-cellular fraction of a body fluid from the subject. The non-cellular fraction can be plasma, serum, or other non-cellular body fluid. In one embodiment, the sample is a serum sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). For example, the sample can be a fine needle biopsy sample, an archival sample (e.g., an archived sample with a known diagnosis and/or treatment history), a histological section (e.g., a frozen or formalin-fixed section, e.g., after long term storage), among others. A sample can include any material obtained and/or derived from a biological sample, including a polypeptide, and nucleic acid (e.g., genomic DNA, cDNA, RNA) purified or processed from the sample. Purification and/or processing of the sample can include one or more of extraction, concentration, antibody isolation, sorting, concentration, fixation, addition of reagents and the like. In one embodiment, the quality and/or integrity of the sample, e.g., a frozen sample, is evaluated by detecting one or more of: a panel of serum markers, e.g., the panel of serum markers (including, e.g., IL-23, IL-15, IL-7, IL-1α, IL-1β, IL-1RA, IFNγ, IL-2-6, IL-8, IL-10, IL-12p40, IL-12p70, IL-15, AAT, A2M, B2M, BDNF, CRP, C3, CCL11, F7, FT, FGA, GM-CSF, HB, ICAM-1M MIP-1a, MIP-1b, MMP2, MMP3, MMP9, CCL2, RANTES, SCF, TIMP, TNFα, TNFβ, TNF-ra2, VCAM-1, VEGF, VWF, VDBP; a selection of these serum markes is shown in FIG. 2, or a subset thereof); evaluating the serum profile by comparing a sample from a control healthy volunteer to a sample from an MS patient, as shown in FIG. 3; evaluating an interferon response signature by detecting one or more of the serum proteins listed in FIG. 4A; or detecting a dose dependent correlation of an interferon signature response marker, e.g., CXCL10, as shown in FIG. 4B. In one embodiment, the sample contains one or more MS biomarkers described herein, e.g., one or more genes or gene products (e.g., cDNA, RNA (e.g., mRNA), or a polypeptide) for the MS biomarkers described herein.

In certain embodiments, the detection or determining steps of the methods or assays described herein include determining quantitatively the value (e.g., level) (e.g., amount or concentration) of an MS biomarker (e.g., one or more of the MS biomarkers described herein) from a sample, e.g., a sample of plasma, serum, or other non-cellular body fluid; or a cellular sample (e.g., a PBMC sample), wherein the amount or concentration of the MS biomarker, thereby provides a value (also referred to herein as a “determined,” or “detected,” “value”). In certain embodiments, the determined or detected value is compared to a specified parameter (e.g., a reference value; a control sample; a sample obtained from a healthy subject; or a sample obtained from the subject at different time intervals, e.g., prior to, during, or after treatment), to thereby diagnose, evaluate, identify a patient, or monitor treatment efficacy or a susceptibility thereto, and/or monitor response to an MS therapy in an individual. In alternative embodiments, the sample is assayed for qualitative, or both quantitative and qualitative determination of the MS biomarker level. In certain embodiments, methods or assays of the invention relate to determining quantitatively the amount or concentration of the MS biomarker from plasma or serum of the subject, wherein the plasma or serum is obtained from the blood of the subject, for example.

In certain embodiments of the methods or assays, an increase in the value (e.g., level) of the MS biomarker relative to a reference value (e.g., a relative or absolute reference value compared to a value from a normal sample, or a non-responder sample) is indicative of increased responsiveness to an MS therapy (e.g., an IFN-b therapy). In embodiments where the MS biomarker is a polypeptide, an increase in the level of one or more of CCL21, BAFF, IL-1RA, MCP-1, CRP, TNFR2 or CXCL10, polypeptude relative to a reference value (e.g., a value from a normal sample, or a non-responder sample) in indicative of increased responsiveness of an MS patient to IFN-b therapy. Exemplary reference values to categorize responders and non-responders are shown in Tables 1-2 herein.

In one embodiment, a value (e.g., level) of CCL21 in the serum equal to, or higher than, about 0.6 or 0.85 ng/ml is indicative of increased responsiveness of an MS subject to an IFN-b therapy, whereas a CCL21 serum level of less than about 0.6 or 0.4 ng/ml is indicative of decreased responsiveness. For example, a value of about 0.7 to 0.85 ng/ml, or about 0.75 to 0.8 ng/ml of CCL21 in the serum of an MS patient is indicative of increased responsiveness of an MS patient to IFN-b therapy; whereas a value of about 0.55 to 0.4 ng/ml, or 0.5 ng/ml of CCL21 in the serum of an MS patient is indicative of decreased responsiveness of an MS patient to IFN-b therapy.

In another embodiment, a value (e.g., level) of BAFF in the serum equal to, or higher than, about 0.95 or 1.10 ng/ml is indicative of increased responsiveness of an MS subject to an IFN-b therapy, whereas a BAFF serum level of less than about 0.95 or 0.8 ng/ml is indicative of decreased responsiveness. For example, a BAFF serum value of about 1.10 to 0.95 ng/ml, or about 1.05 to 1.0 ng/ml of in the serum of an MS patient is indicative of increased responsiveness of an MS patient to IFN-b therapy; whereas a value of about 0.93 to 0.8 ng/ml, or about 0.9 ng/ml of BAFF in the serum of an MS patient is indicative of decreased responsiveness of an MS patient to IFN-b therapy.

In one embodiment, a value (e.g., level) of IL-1RA in the serum equal to, or higher than, about 0.12 or 0.2 ng/ml is indicative of increased responsiveness of an MS subject to an IFN-b therapy, whereas an IL-1RA serum level of less than about 0.12 or 0.05 ng/ml is indicative of decreased responsiveness. For example, an IL-1RA serum value of about 0.2 to 0.12 ng/ml, or about 0.15 to 0.12 ng/ml of in the serum of an MS patient is indicative of increased responsiveness of an MS patient to IFN-b therapy; whereas a value of about 0.10 to 0.05 ng/ml, or about 0.09 to 0.08 ng/ml of IL-1RA in the serum of an MS patient is indicative of decreased responsiveness of an MS patient to IFN-b therapy.

In one embodiment, a value (e.g., level) of MCP-1 in the serum equal to, or higher than, about 0.45 or 0.55 ng/ml is indicative of increased responsiveness of an MS subject to an IFN-b therapy, whereas an MCP-1 serum level of less than about 0.45 or 0.40 ng/ml is indicative of decreased responsiveness. For example, an MCP-1 serum value of about 0.55 to 0.45 ng/ml, or about 0.50 to 0.48 ng/m of in the serum of an MS patient is indicative of increased responsiveness of an MS patient to IFN-b therapy; whereas a value of about 0.44 to 0.40 ng/ml, or about 0.42 to 0.41 ng/ml of MCP-1 in the serum of an MS patient is indicative of decreased responsiveness of an MS patient to IFN-b therapy.

In one embodiment, a value (e.g., level) of CRP in the serum equal to, or higher than, about 0.0015 or 0.0025 ng/ml is indicative of increased responsiveness of an MS subject to an IFN-b therapy, whereas a CRP serum level of less than about 0.0015 or 0.0008 ng/ml is indicative of decreased responsiveness. For example, a CRP serum value of about 0.0025 to 0.0015 ng/ml, or about 0.0020 to 0.0018 ng/ml of in the serum of an MS patient is indicative of increased responsiveness of an MS patient to IFN-b therapy; whereas a value of about 0.0014 to 0.0008 ng/ml, or about 0.0012 to 0.0010 ng/ml of CRP in the serum of an MS patient is indicative of decreased responsiveness of an MS patient to IFN-b therapy.

In one embodiment, a value (e.g., level) of B2M in the serum equal to, or higher than, about 0.0014 or 0.0025 ng/ml is indicative of increased responsiveness of an MS subject to an IFN-b therapy, whereas a B2M serum level of less than about 0.0014 or 0.0009 ng/ml is indicative of decreased responsiveness. For example, a B2M serum value of about 0.0025 to 0.0014 ng/ml, or about 0.0020 to 0.0015 ng/ml of in the serum of an MS patient is indicative of increased responsiveness of an MS patient to IFN-b therapy; whereas a value of about 0.0013 to 0.0009 ng/ml, or about 0.0013 to 0.0010 ng/ml of B2M in the serum of an MS patient is indicative of decreased responsiveness of an MS patient to IFN-b therapy.

In one embodiment, a value (e.g., level) of TNFR2 in the serum equal to, or higher than, about 0.005 or 0.006 ng/ml is indicative of increased responsiveness of an MS subject to an IFN-b therapy, whereas a TNFR2 serum level of less than about 0.005 or 0.004 ng/ml is indicative of decreased responsiveness. For example, a TNFR2 serum value of about 0.006 to 0.005 ng/ml, or about 0.0055 to 0.0052 ng/ml of in the serum of an MS patient is indicative of increased responsiveness of an MS patient to IFN-b therapy; whereas a value of about 0.0048 to 0.0035 ng/ml, or about 0.0045 to 0.004 ng/ml of TNFR2 in the serum of an MS patient is indicative of decreased responsiveness of an MS patient to IFN-b therapy. In other embodiments, a decrease in the level of the MS biomarker relative to a reference value (e.g., a value from a normal sample, or a non-responder sample) is indicative of increased responsiveness to an MS therapy (e.g., an IFN-b therapy).

In embodiments where the MS biomarker is a polypeptide, a decrease in the value (e.g., level) of IL-13 or ferritin polypeptide, relative to a reference value (e.g., a value from a normal sample, or a non-responder sample) in indicative of increased responsiveness of an MS patient to IFN-b therapy. In one embodiment, a level of IL-13 in the serum equal to, or less than, about 0.01 or 0.001 ng/ml is indicative of increased responsiveness of an MS subject to an IFN-b therapy, whereas an IL-13 serum level greater than about 0.01 or 0.035 ng/ml is indicative of decreased responsiveness. For example, an IL-13 serum value of about 0.001 to 0.01 ng/ml, or about 0.006 to 0.008 ng/m of in the serum of an MS patient is indicative of increased responsiveness of an MS patient to IFN-b therapy; whereas a value of about 0.011 to 0.035 ng/ml, or about 0.025 to 0.030 ng/ml of IL-13 in the serum of an MS patient is indicative of decreased responsiveness of an MS patient to IFN-b therapy.

In one embodiment, the method or assay includes comparing the value (e.g., level) of one or more MS biomarkers to a specified parameter (e.g., a reference value or sample; a sample obtained from a healthy subject; a sample obtained from a patient at different treatment intervals). For example, a sample can be analyzed at any stage of treatment, but preferably, prior to, during, or after terminating, administration of the MS therapy, to thereby determine appropriate dosage(s) and treatment regimen(s) of the MS therapy (e.g., amount per treatment or frequency of treatments) for prophylactic or therapeutic treatment of the subject. In certain embodiments, the methods, or assays, of the invention include the step of detecting the level of one or more MS biomarkers in the subject, prior to, or after, administering the MS therapy, to the subject. A level of the MS biomarker in the range of responsiveness described herein in the sample (e.g., a serum sample) indicates that the subject from whom the sample was obtained is likely to show IFN-b responsiveness. A level of the MS biomarker in the range of non-responsiveness described herein in the sample (e.g., a serum sample) indicates that the subject from whom the sample was obtained is unlikely to show IFN-b responsiveness, and thus, alternative MS therapies can be considered, including, but not limited to, glatiramer (COPAXONE®), natalizumab (TYSABRI®), mitoxantrone (NOVANTRONE®), fingolimod (FTY720; GILENYA®), dimethyl fumarate (e.g., an oral dimethyl fumarate (BG-12)), Daclizumab, alemtuzumab (LEMTRADA®)), or an anti-LINGO-1 antibody.

In certain embodiments, the MS biomarker evaluated is a gene or gene product, e.g., cDNA, RNA (e.g., mRNA), or a polypeptide. In embodiments where the MS biomarker is a polypeptide, the polypeptide can be detected, or the level determined, by any means of polypeptide detection, or detection of the expression level of the polypeptides. For example, the polypeptide can be detected using a reagent which specifically binds with the MS biomarker polypeptides. In another embodiment, the reagent is selected from the group consisting of an antibody, an antibody derivative, and an antibody fragment. In one embodiment, the MS biomarker is detected using antibody-based detection techniques, such as enzyme-based immunoabsorbent assay, immunofluorescence cell sorting (FACS), immunohistochemistry, immunofluorescence (IF), antigen retrieval and/or microarray detection methods. In one embodiment, the detection, or determination of the level, of the MS biomarker includes contacting the sample with a reagent, e.g., an antibody that binds to the MS biomarker and detecting or determining the level of the reagent, e.g., the antibody, bound to the MS biomarker. The reagent, e.g., the antibody, can be labeled with a detectable label (e.g., a fluorescent or a radioactive label). Polypeptide detection methods can be performed in any other assay format, including but not limited to, ELISA, RIA, and mass spectrometry. The amount, structure and/or activity of the MS biomarker polypeptides can be compared to a reference value, e.g., a control sample, or a pre-determined value. In one embodiment, the detection or determination step includes a multiplex bead enzyme-based immunoabsorbent assay. In such embodiments, the detection is usually driven by a fluorescent molecule bound to the detection antibody by biotin.

In other embodiments where the MS biomarker is a nucleic acid, the nucleic acid can be detected, or the level determined, by any means of nucleic acid detection, or detection of the expression level of the nucleic acids, including but not limited to, nucleic acid hybridization assay, amplification-based assays (e.g., polymerase chain reaction), sequencing, screening analysis (including metaphase cytogenetic analysis by standard karyotype methods, FISH, spectral karyotyping or MFISH, and comparative genomic hybridization), and/or in situ hybridization. The amount, structure and/or activity of the one or more MS biomarker nucleic acid (e.g., DNA or RNA) can be compared to a reference value or sample, e.g., a control sample, or a pre-determined value.

In yet another embodiment, the one or more MS biomarkers are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to worsening disease. In another embodiment, the significant event is the progression from primary diagnosis to relapse. In another embodiment, the significant event is the progression from secondary MS to death. In another embodiment, the significant event is the progression from remission to relapse. In another embodiment, the significant event is the progression from relapse to death. In certain embodiments, the time course is measured with respect to one or more overall survival rate, time to progression and/or using the EDSS or other assessment criteria.

In one embodiment, the one or more MS biomarkers are assessed in an MS patient (e.g., a patient with RRMS) prior to administration of an MS therapy described herein (e.g., prior to administration of an IFN-b agent). In one embodiment, the one or more MS biomarkers are assessed in a newly diagnosed RRMS patient, e.g., a newly diagnosed RRMS patient prior to IFN-b therapy. In another embodiment, the one or more MS biomarkers are assessed in an MS patient (e.g., an RRMS patient) after administration of an MS therapy described herein (e.g., IFN-b agent) (e.g., after administration of the MS therapy for one, two weeks, one month, two months, three months, four months, six months, one year or more).

In certain embodiments, a pre-determined measure or value is created after evaluating the sample by dividing subject's samples into at least two patient subgroups (e.g., responders vs. non-responders). In certain embodiments, the number of subgroups is two, such that the patient sample is divided into a subgroup of patients having a specified level of the one or more MS biomarkers described herein, and a subgroup not having the specified level of the one or more MS biomarkers. In certain embodiments, the MS biomarker status in the subject is compared to either the subgroup having or not having the specified level of the one or more MS biomarker, if the MS patient has a specified value, e.g., a level of the MS biomarker, in the range of responsiveness described herein in the sample (e.g., a serum sample), then the MS patient is likely to respond to IFN-b 1b therapy; alternatively, if the MS patient has a specified value, e.g., a level of the MS biomarker, in the range of non-responsiveness described herein in the sample (e.g., a serum sample), then the MS patient is unlikely to respond to IFN-b 1b therapy. In certain embodiments, the number of subgroups is greater than two, including, without limitation, three subgroups, four subgroups, five subgroups and six subgroups, depending on stratification of predicted IFN-b 1b therapy efficacy as correlated with particular MS biomarkers.

Alternatively, or in combination with the methods described herein, the invention features a method of treating, or preventing in, a subject having multiple sclerosis (MS) one or more symptoms associated with MS. In one embodiment, the subject is identified as likely or unlikely to respond to IFN-b 1a therapy, using the methods, or assays, described herein. In certain embodiments, the treatment includes reducing, retarding or preventing, a relapse, or the worsening of a disability, in the MS patients. In one embodiment, the method includes administering to a subject (e.g., a patient with RRMS) a therapy for MS (also referred to herein as an “MS therapy”), e.g., disease modifying MS therapy, in an amount sufficient to reduce one or more symptoms associated with MS. In one embodiment, the MS therapy includes an IFN-b agent (e.g., an IFN-b 1a molecule or an IFN-b 1b molecule, including analogues and derivatives thereof (e.g., pegylated variants thereof)). In one embodiment, the MS therapy includes an IFN-b 1a agent (e.g., AVONEX®, REBIF®). In another embodiment, the MS therapy includes an INF-b 1b agent (e.g., BETASERON®, Betaferon®). In another embodiment where the IFN-1b therapy is unlikely to be effective (e.g., by identifying the subject as unlikely to be responsive to IFN-b 1b therapy), the MS therapy chosen can be an alternative MS therapy, e.g., a therapy that includes a polymer of four amino acids found in myelin basic protein, e.g., a polymer of glutamic acid, lysine, alanine and tyrosine (e.g., glatiramer (COPAXONE®)); an antibody or fragment thereof against alpha-4 integrin (e.g., natalizumab (TYSABRI®)); an anthracenedione molecule (e.g., mitoxantrone (NOVANTRONE®)); or fingolimod (FTY720; GILENYA®). In certain embodiments, the methods include the use of one or more symptom management therapies, such as antidepressants, analgesics, anti-tremor agents, among others.

In other embodiments, the methods, assays, and/or kits described herein further include providing or generating, and/or transmitting information, e.g., a report, containing data of the evaluation or treatment determined by the methods, assays, and/or kits as described herein. The information can be transmitted to a report-receiving party or entity (e.g., a patient, a health care provider, a diagnostic provider, and/or a regulatory agency, e.g., the FDA), or otherwise submitting information about the methods, assays and kits disclosed herein to another party. The method can relate to compliance with a regulatory requirement, e.g., a pre- or post approval requirement of a regulatory agency, e.g., the FDA. In one embodiment, the report-receiving party or entity can determine if a predetermined requirement or reference value is met by the data, and, optionally, a response from the report-receiving entity or party is received, e.g., by a physician, patient, diagnostic provider.

In another aspect, the invention features a method of treating a patient having MS or at risk for developing MS. The method includes: (optionally) (a) providing or collecting a sample from a subject, e.g., a sample and a subject as described herein; (b) evaluating the sample to detect, or determine the level, of one or more MS biomarkers as described herein; and (c) administering to said subject a therapeutically effective amount of an MS therapy, e.g., disease modifying MS therapy, in an amount sufficient to reduce one or more symptoms associated with MS. In one embodiment, the MS therapy includes an IFNb agent (e.g., an IFN-b 1a molecule or an IFN-b 1b molecule, including analogues and derivatives thereof (e.g., pegylated variants thereof)). In one embodiment, the MS therapy includes an IFN-b 1a agent (e.g., AVONEX®, REBIF®). In another embodiment, the MS therapy includes an INFb-1b agent (e.g., BETASERON®, BETAFERON®). In another embodiment where IFN-b 1b therapy is unlikely to be effective (e.g., by identifying the subject as unlikely to be responsive to IFN-b 1b therapy), the MS therapy chosen can be an alternative MS therapy, e.g., an MS therapy chosen can be an alternative MS therapy, e.g., a therapy that includes a polymer of four amino acids found in myelin basic protein, e.g., a polymer of glutamic acid, lysine, alanine and tyrosine (e.g., glatiramer (COPAXONE®)); an antibody or fragment thereof against alpha-4 integrin (e.g., natalizumab (TYSABRI®)); an anthracenedione molecule (e.g., mitoxantrone (NOVANTRONE®)); or fingolimod (FTY720; GILENYA®); a dimethyl fumarate (e.g., an oral dimethyl fumarate (BG-12)); an antibody to the alpha subunit of the IL-2 receptor of T cells (e.g., Daclizumab); an antibody against CD52 (e.g., alemtuzumab (LEMTRADA®)); or an anti-LINGO-1 antibody. In certain embodiments, the methods include the use of one or more symptom management therapies, such as antidepressants, analgesics, anti-tremor agents, among others.

The methods of the invention can further include the step of monitoring the subject, e.g., for a change (e.g., an increase or decrease) in one or more of: levels of one or more MS biomarkers; the rate of appearance of new lesions, e.g., in an MRI scan; the appearance of new disease-related symptoms; a change in EDSS score; a change in quality of life; or any other parameter related to clinical outcome. The subject can be monitored in one or more of the following periods: prior to beginning of treatment; during the treatment; or after the treatment has been administered. Monitoring can be used to evaluate the need for further treatment with the same MS therapy, or for additional MS treatment. Generally, a decrease in one or more of the parameters described above is indicative of the improved condition of the subject.

In another aspect, the invention features kits for evaluating a sample, e.g., a sample from an MS patient, to detect or determine the level of one or more MS biomarkers. The kit includes a means for detection of (e.g., a reagent that specifically detects) one or more MS biomarkers as described herein. In certain embodiments, the kit includes an MS therapy. In one another embodiment, the kit comprises an antibody, an antibody derivative, or an antibody fragment to an MS biomarker polypeptide. In one embodiment, the kit includes an antibody-based detection technique, such as immunofluorescence cell sorting (FACS), immunohistochemistry, antigen retrieval and/or microarray detection reagents. In one embodiment, at least one of the reagents in the kit is an antibody that binds to an MS biomarker (optionally) with a detectable label (e.g., a fluorescent or a radioactive label). In certain embodiments, the kit is an ELISA or an immunohistochemistry (IHC) assay for detection of the MS biomarker.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE FIGURES

FIG. 1A is a schematic representation summarizing the retrospective biomarker study.

FIGS. 1B-1C is a set of bar graphs depicting the frequency of new or enlarging T2 lesions in a subset of patients that underwent an MRI assessment of lesions. For the 118 Responder and Non-Responder patients, 40 subjects had measurements of New Enlarging T2 lesions for 3 years (40/118=34%).

FIG. 2 is a graph depicting the concentration of 35 different analytes in multiple sclerosis patients prior to treatment with Avonex® (MS-PRE) and in healthy volunteers (HV) to confirm that the sample quality of the stored MS-PRE sera was acceptable for further analysis.

FIG. 3 is a table depicting differences in analyte protein expression of markers in serum from multiple sclerosis patients prior to treatment with Avonex® (MS-PRE) as compared to expression of the markers in the serum of healthy volunteers (HV). The data in FIG. 3 follow expected literature values and confirm that the stored serum samples were not degraded.

FIGS. 4A-4B show that CXCL10 expression and expected use as a biomarker for multiple sclerosis was confirmed, thus indicating that the stored samples were not degraded. The P-values were from tests on the ration of 3 month and baseline between 30 μg and 60 μg.

FIGS. 5A-5C show expression data for the biomarkers CCL21, BAFF, CRP, and IL-1RA in both non-responders and responders at baseline and 3-months after treatment with Avonex®.

FIG. 6 is a table showing the analysis of the MRI subset for predictive biomarkers and further shows that the expression of the CCL21 and BAFF biomarkers were significant.

FIGS. 7A-7E depics a series of graphs depicting the expression of CCL21, BAFF, IL-1RA, MCP-1, and TNFRII expression in non-responders and responders at baseline.

FIGS. 8A-8B shows the sensitivity, specificity, and AUC for CCL21 and BAFF as predictors of R/NR classification within an MRI subset.

FIGS. 9A-9C show data identifying IL-13 as a biomarker to classify responders vs. non-responders.

FIGS. 10A-10B are tables showing the unadjusted p-values for a list of potential biomarkers in B1 (general population of R/NR; n=118) and B2 (MRI subset n=30).

FIGS. 11A-11B show levels of the biomarker ferritin in non-responders vs. responders separated by age groups at baseline and 3 months after treatment initiation.

### DETAILED DESCRIPTION OF THE INVENTION

Methods, assays and kits for the identification, assessment and/or treatment of a subject having multiple sclerosis (MS) (e.g., a patient with relapsing-remitting multiple sclerosis (RRMS)) are disclosed. In one embodiment, responsiveness of a subject to an interferon beta (“IFN-β” or “IFN-b”) agent (e.g., an IFN-β 1a molecule or an IFN-β 1b molecule) is determined by evaluating an alteration (e.g., an increased or decreased level) of an MS biomarker in a sample, e.g., a serum sample obtained from an MS patient. In certain embodiments, the MS biomarker evaluated CCL21 and/or BAFF, and one or more of IL-1RA, IL-13, MCP-1, CRP, B2M, ferritin, and/or TNFR2.

In one embodiment, serum levels of CCL21 and BAFF were shown to classify MS patients with RRMS who are responders and nonresponders to IFNbeta-1a, when using a highly restrictive measure of responders and non-responders, which included a combination of EDSS, relapse and MRI parameters of three years. Thus, the invention can, therefore, be used as a means to evaluate responsiveness to, or monitor, a therapy, e.g., an MS therapy (e.g., an MS therapy that includes an IFN-b agent); identify a patient as likely to benefit from such agents; stratify patient populations (e.g., stratify patients as likely or unlikely to respond (e.g., responders vs. non-responders) to a therapy, e.g., an MS therapy (e.g., an MS therapy that includes an IFN-b agent); and/or more effectively monitor, treat multiple sclerosis or prevent worsening of disease and/or relapse.

Various aspects of the invention are described in further detail in the following subsections.

### DEFINITIONS

As used herein, each of the following terms has the meaning associated with it in this section.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

The term “altered level of expression” of a biomarker as described herein (e.g., CCL21, BAFF, IL-1RA, IL-13, MCP-1, CRP, B2M, ferritin, and TNFR2) refers to an increase (or decrease) in the expression level of a marker in a test sample, such as a sample derived from a patient suffering from multiple sclerosis or a similar disorder (e.g., clinically isolated syndrome (CIS), benign MS), that is greater or less than the standard error of the assay employed to assess expression. In embodiments, the alteration can be at least twice, at least twice three, at least twice four, at least twice five, or at least twice ten or more times greater than or less than the expression level of the biomarkers in a control sample (e.g., a sample from a healthy subject not having the associated disease), or the average expression level in several control samples. An “altered level of expression” can be determined at the protein or nucleic acid (e.g., mRNA) level.

“Binding compound” shall refer to a binding composition, such as a small molecule, an antibody, a peptide, a peptide or non-peptide ligand, a protein, an oligonucleotide, an oligonucleotide analog, such as a peptide nucleic acid, a lectin, or any other molecular entity that is capable of specifically binding to a target protein or molecule or stable complex formation with an analyte of interest, such as a complex of proteins.

“Binding moiety” means any molecule to which molecular tags can be directly or indirectly attached that is capable of specifically binding to an analyte. Binding moieties include, but are not limited to, antibodies, antibody binding compositions, peptides, proteins, nucleic acids and organic molecules having a molecular weight of up to about 1000 daltons and containing atoms selected from the group consisting of hydrogen, carbon, oxygen, nitrogen, sulfur and phosphorus.

A “biomarker” or “marker” is a gene, mRNA, or protein that undergoes alterations in expression that are associated with multiple sclerosis or responsiveness to treatment with IFN-β. The alteration can be in amount and/or activity in a biological sample (e.g., a blood, plasma, or a serum sample) obtained from a subject having multiple sclerosis, as compared to its amount and/or activity, in a biological sample obtained from a healthy subject (e.g., a control); such alterations in expression and/or activity are associated with a disease state, such as multiple sclerosis. For example, a marker of the invention which is associated with multiple sclerosis or predictive of responsiveness to IFN-β therapeutics can have an altered expression level, protein level, or protein activity, in a biological sample obtained from a subject having, or suspected of having, multiple sclerosis as compared to a biological sample obtained from a control subject (e.g., a healthy individual).

A “nucleic acid” “marker” or “biomarker” is a nucleic acid (e.g., DNA, mRNA, cDNA) encoded by or corresponding to a marker as described herein. For example, such marker nucleic acid molecules include DNA (e.g., genomic DNA and cDNA) comprising the entire or a partial sequence of any of the nucleic acid sequences set forth herein (e.g., in Table 1), or the complement or hybridizing fragment of such a sequence. The marker nucleic acid molecules also include RNA comprising the entire or a partial sequence of any of the nucleic acid sequences set forth herein (e.g., in Table 1), or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth herein (e.g., in Table 1), or a fragment thereof. The terms “protein” and “polypeptide” are used interchangeably herein.

A marker is “fixed” to a substrate if it is covalently or non-covalently associated with the substrate such that the substrate can be rinsed with a fluid (e.g., standard saline citrate, pH 7.4) without a substantial fraction of the marker dissociating from the substrate.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, at least 60%, at least 65%, at least 70%, at least 75%, at least 80%, at least 85%, at least 90%, at least 95% or more.

Multiple sclerosis is “treated,” “inhibited” or “reduced,” if at least one symptom of the disease is reduced, alleviated, terminated, slowed, or prevented. As used herein, multiple sclerosis is also “treated,” “inhibited,” or “reduced,” if recurrence or relapse of the disease is reduced, slowed, delayed, or prevented. Exemplary clinical symptoms of multiple sclerosis that can be used to aid in determining the disease status in a subject can include e.g., tingling, numbness, muscle weakness, loss of balance, blurred or double vision, slurred speech, sudden onset paralysis, lack of coordination, cognitive difficulties, fatigue, heat sensitivity, spasticity, dizziness, tremors, gait abnormalities, speech/swallowing difficulties, and extent of lesions assessed by imaging techniques, e.g., MRI. Clinical symptoms of MS are routinely classified and standardized, e.g., using an EDSS rating system. Typically, a decrease of one full step indicates an effective MS treatment (Kurtzke, Ann. Neurol. 36:573-79, 1994), while an increase of one full step will indicate the progression or worsening of the disease (e.g., exacerbation).

The terms “therapy” or “treatment” (e.g., MS therapy or MS treatment) are used interchangeably herein.

As used herein, the “Expanded Disability Status Scale” or “EDSS” is intended to have its customary meaning in the medical practice. EDSS is a rating system that is frequently used for classifying and standardizing MS. The accepted scores range from 0 (normal) to 10 (death due to MS). Typically patients having an EDSS score of about 6 will have moderate disability (e.g., walk with a cane), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability imparing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

An “overexpression” or “significantly higher level of expression” of the gene products (e.g., the markers set forth in Table 1) refers to an expression level or copy number in a test sample that is greater than the standard error of the assay employed to assess the level of expression. In embodiments, the overexpression can be at least two, at least three, at least four, at least five, or at least ten or more times the expression level of the gene products (e.g., the markers set forth in Table 1) in a control sample (e.g., a sample from a healthy subject not afflicted with multiple sclerosis), or the average expression level of gene products (e.g., the markers set forth in Table 1) in several control samples.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

“Responsiveness,” to “respond” to treatment, and other forms of this verb, as used herein, refer to the reaction of a subject to treatment with an MS therapy, e.g., a therapy including an IFN-β agent. As an example, a subject responds to treatment with an IFN-β agent if at least one symptom of multiple sclerosis (e.g., relapse rate) in the subject is reduced or retarded by about 10%, 20%, 30%, 40%, 50%, 60%, 70%, 80%, 90% or more. In another example, a subject responds to treatment with an IFN-β agent, if at least one symptom of multiple sclerosis in the subject is reduced by about 5%, 10%, 20%, 30%, 40%, 50% or more as determined by any appropriate measure, e.g., Expanded Disability Status Scale (EDSS) or determining the extent of other symptoms such as relapse rate, muscle weakness, tingling, and numbness. In another example, a subject responds to treatment with an IFN-β agent, if the subject experiences a life expectancy extended by about 5%, 10%, 20%, 30%, 40%, 50% or more beyond the life expectancy predicted if no treatment is administered. In another example, a subject responds to treatment with an IFN-β agent, if the subject has an increased disease-free survival, overall survival or increased time to progression. Several methods can be used to determine if a patient responds to a treatment including the EDSS criteria, as set forth above.

A “responder” refers to a subject, e.g., an MS patient, if in response to an MS therapy (e.g., IFN beta therapy), at least one symptom of multiple sclerosis in the subject is reduced by about 5%, 10%, 20%, 30%, 40%, 50% or more as determined by any appropriate measure, e.g., EDSS or determining the extent of other symptoms such as relapse rate, muscle weakness, tingling, and numbness. In one embodiment, a responder is defined as a subject with no confirmed relapses and no evidence of sustained disability progression (by EDSS) during the first three years of treatment (e.g., clinical remission).

A “non-responder” refers to a subject, e.g., an MS patient, if in response to an MS therapy (e.g., IFN beta therapy), at least one symptom of multiple sclerosis in the subject is reduced by less than about 5%, as determined by any appropriate measure, e.g., EDSS or determining the extent of other symptoms such as relapse rate, muscle weakness, tingling, and numbness. In one embodiment, a non-responder is defined as those subjects that have active disease on therapy including subjects with at least 3 relapses, development of a 6-month sustained progression in disability defined as a 1.0 point increase in EDSS score from baseline in subjects with a baseline score of ≦5.5. Subjects were excluded for having ≧10 MRI T2 lesions in the remission or permanently testing positive for NAB starting from year 1 at any titer or NAB titers ≧20 in either group.

“Likely to” or “increased likelihood,” as used herein, refers to an increased probability that an item, object, thing or person will occur. Thus, in one example, a subject that is likely to respond to treatment with an IFN-β agent to treat multiple sclerosis has an increased probability of responding to treatment with an IFN-β agent to treat multiple sclerosis, relative to a reference subject or group of subjects.

“Unlikely to” refers to a decreased probability that an event, item, object, thing or person will occur with respect to a reference. Thus, a subject that is unlikely to respond to treatment with an IFN-β agent has a decreased probability of responding to treatment with an IFN-β agent relative to a reference subject or group of subjects.

“Sample,” “tissue sample,” “patient sample,” “patient cell or tissue sample” or “specimen” each refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bodily fluids such as cerebral spinal fluid, whole blood, plasma and serum. The sample can include a non-cellular fraction (e.g., plasma, serum, or other non-cellular body fluid). In one embodiment, the sample is a serum sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

The amount of a biomarker, e.g., expression of gene products (e.g., one or more the biomarkers described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about 1.5, two, at least about three, at least about four, or at least about five times, higher or lower, respectively, than the normal amount of the marker.

As used herein, “significant event” shall refer to an event in a patient's disease that is important as determined by one skilled in the art. Examples of significant events include, for example, without limitation, primary diagnosis, death, recurrence, remission, relapse of a patient's disease or the progression of a patient's disease from any one of the above noted stages to another. A significant event can be any important event used determine disease status using e.g., EDSS or other symptom criteria, as determined by one skilled in the art.

As used herein, “time course” shall refer to the amount of time between an initial event and a subsequent event. For example, with respect to a patient's disease, time course can relate to a patient's disease and can be measured by gauging significant events in the course of the disease, wherein the first event can be diagnosis and the subsequent event can be remission or relapse, for example.

A “transcribed polynucleotide” is a polynucleotide (e.g., an RNA, a cDNA, or an analog of one of an RNA or cDNA) which is complementary to or homologous with all or a portion of a mature RNA made by transcription of a marker of the invention and normal post-transcriptional processing (e.g., splicing), if any, of the transcript, and reverse transcription of the transcript.

An “underexpression” or “significantly lower level of expression” of products (e.g., the markers set forth herein) refers to an expression level in a test sample that is greater than the standard error of the assay employed to assess expression, for example, at least 1.5, twice, at least three, at least four, at least five, or at least ten or more times less than the expression level of the gene products (e.g., the markers set forth in Table 1) in a control sample (e.g., a sample from a healthy subject not afflicted with multiple sclerosis), or the average expression level of gene products (e.g., the markers set forth in Table 1) in several control samples.

Various aspects of the invention are described in further detail below. Additional definitions are set out throughout the specification.

### Multiple Sclerosis and Methods of Diagnosis

Multiple sclerosis (MS) is a central nervous system disease that is characterized by inflammation and loss of myelin sheaths.

Patients having MS can be identified by clinical criteria establishing a diagnosis of clinically definite MS as defined by Poser et al., Ann. Neurol. 13:227, 1983. Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for Multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks. Effective treatment of multiple sclerosis may be evaluated in several different ways. The following parameters can be used to gauge effectiveness of treatment. Two exemplary criteria include: EDSS (extended disability status scale), and appearance of exacerbations on MRI (magnetic resonance imaging).

The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step indicates an effective treatment (Kurtzke, Ann. Neurol. 36:573-79, 1994), while an increase of one full step will indicate the progression or worsening of disease (e.g., exacerbation). Typically patients having an EDSS score of about 6 will have moderate disability (e.g., walk with a cane), whereas patients having an EDSS score of about 7 or 8 will have severe disability (e.g., will require a wheelchair).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are mild, moderate, or severe according to changes in a Neurological Rating Scale (Sipe et al., Neurology 34:1368, 1984). An annual exacerbation rate and proportion of exacerbation-free patients are determined.

Therapy can be deemed to be effective using a clinical measure if there is a statistically significant difference in the rate or proportion of exacerbation-free or relapse-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group. An exacerbation-free or relapse-free period of greater than one year, 18 months, or 20 months is particularly noteworthy. Clinical measurements include the relapse rate in one and two-year intervals, and a change in EDSS, including time to progression from baseline of 1.0 unit on the EDSS that persists for six months. On a Kaplan-Meier curve, a delay in sustained progression of disability shows efficacy. Other criteria include a change in area and volume of T2 images on MRI, and the number and volume of lesions determined by gadolinium enhanced images.

MRI can be used to measure active lesions using gadolinium-DTPA-enhanced imaging (McDonald et al., Ann. Neurol. 36:14, 1994) or the location and extent of lesions using T2-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, percentage change in lesion area (Paty et al., Neurology 43:665, 1993). Improvement due to therapy can be established by a statistically significant improvement in an individual patient compared to baseline or in a treated group versus a placebo group.

Exemplary symptoms associated with multiple sclerosis, which can be treated with the methods described herein or managed using symptom management therapies, include: optic neuritis, diplopia, nystagmus, ocular dysmetria, internuclear opthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, uhthoffs symptom, gastroesophageal reflux, and sleeping disorders.

Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting MS (RRMS). Primary-progressive MS (PPMS) is characterized by a gradual clinical decline with no distinct remissions, although there may be temporary plateaus or minor relief from symptoms. Secondary-progressive MS (SPMS) begins with a relapsing-remitting course followed by a later primary-progressive course. Rarely, patients may have a progressive-relapsing (PRMS) course in which the disease takes a progressive path punctuated by acute attacks. PPMS, SPMS, and PRMS are sometimes lumped together and called chronic progressive MS.

A few patients experience malignant MS, defined as a swift and relentless decline resulting in significant disability or even death shortly after disease onset. This decline may be arrested or decelerated by determining the likelihood of the patient to respond to a therapy early in the therapeutic regime and switching the patient to an agent that they have the highest likelihood of responding to.

### Analysis of MS Biomarkers

Analysis of levels of expression and/or activity of gene products in the IFN-β signaling pathway has led to the identification of individual biomarkers and combinations of biomarkers described herein, which correlate with the efficacy of IFN-β agents, alone or in combination, e.g., in combination with another agent for treating multiple sclerosis, in a subject. For example, the present invention provides methods for evaluation of expression level, protein level, protein activity of e.g., CCL21, BAFF, IL-1RA, IL-13, MCP-1, CRP, B2M, ferritin, and TNFR2.

In some embodiments, methods of the present invention can be used to determine the responsiveness of a subject to treatment with an IFN-β agent (e.g., an IFNβ-1A, an IFNβ-1B, or a derivative thereof (e.g., a PEGylated derivative)), wherein if a sample in a subject has a significant increase in the amount, e.g., expression, and/or activity of a marker disclosed herein (e.g., listed in Table 1) relative to a standard, e.g., the level of expression and/or activity in a healthy subject then the disease is more likely to respond to treatment with an the IFN-β agent, alone or in combination with other therapies for multiple sclerosis, and vice versa.

TABLE 1
Serum biomarkers for determining therapeutic response to
IFNβ-1A or IFN β-1B treatment

| | | |
|---|---|---|
| CCL21 = 0.6 ng/ML | MCP-1 = 0.45 ng/ML | CRP = 0.0015 ng/ML |
| BAFF = 0.95 ng/ML | TNFR-2 = 0.005 ng/ML | B2M = 0.0014 ng/ML |
| IL-1RA = 0.12 ng/ML | IL-13 = 0.01 ng/ML | Ferritin = |
| | | (Depends on age |
| | | group) |

TABLE 2
MS Biomarker Protein Levels in Responders (R) vs.
Non-Responders (NR)

| | Protein change in Responders |
|---|---|
| Biomarker | vs. NR |
| CCL21 | Increased |
| | (0.8 ng/mL in R; 0.5 ng/mL in NR) |
| BAFF | Increased |
| | (1.05 ng/mL in R; 0.9 ng/mL in NR) |
| IL-1RA | Increased |
| | (0.14 ng/mL in R; 0.09 ng/mL in NR) |
| MCP-1 | Increased |
| | (0.48 ng/mL in R; 0.42 ng/mL in NR) |
| CRP | Increased |
| | (0.0018 ng/mL in R; 0.0012 ng/mL in NR) |
| B2M | Increased |
| | (0.0015 ng/mL in R; 0.0013 ng/mL in NR) |
| Ferritin | Depends on age group |
| TNFR2 | Increased |
| | (0.0052 ng/mL in R; 0.0045 ng/mL in NR) |
| IL-13 | Decreased |
| | (0.006 ng/mL in R; 0.025 ng/mL in NR) |

The serum biomarkers in Table 1 are described in further detail below.

Chemokine (C-C Motif) Ligand 21 (CCL21):

The nucleotide and protein sequences of human CCL21 are disclosed e.g., in Nagira, M et al. (1997) J. Biol. Chem. 272:19518-19524; Hedrick, J A et al. (1997) J Immunol 159:1589-1593; Hromas, R et al. (1997) J Immunol 159:2554-2558; Gunn, M D et al. (1998) PNAS 95:258-263; Johnson, L A et al. (2010) Int Immunol 22(10):839-849; and Yoshida, R. et al. (1998) J Biol Chem 273(12):7118-7122. CCL21 is highly expressed in high endothelial venules of lymph nodes, spleen and appendix and functions to inhibit hemopoiesis and stimulate chemotaxis of T-cells, particularly naïve T-cells. CCL21 may also play a role in mediating homing of lymphocytes to secondary lymphoid organs. Antibodies for CCL21 are available from a variety of commercial sources including, but not limited to, ABCAM®, ABD SEROTEC™, ABNOVA CORPORATION™, THERMO SCIENTIFIC PIERCE ANTIBODIES™, ACRIS ANTIBODIES™, ANTIGENIX AMERICA™, CELL SCIENCES®, GENETEX™, LIFESPAN BIOSCIENCES™ NOVUS BIOLOGICALS®, R&D SYSTEMS®, SANTA CRUZ BIOTECHNOLOGY® and SIGMA-ALDRICH®.

BAFF (Also Known as TNFSF13B and BLyS):

The nucleotide and protein sequences of human BAFF are disclosed e.g., in Schneider, P et al. (1999) J Exp Med 189:1747-1756; Moore, P A et al. (1999) Science 285:260-263; and Tribouley, C et al. (1999) Biol Chem 380(12):1443-1447. BAFF is a cytokine involved in the stimulation of B- and T-cell function for the regulation of humoral immunity, and promotes the survival of mature B-cells. BAFF is highly expressed in peripheral blood leukocytes and in monocytes and macrophages. BAFF is also expressed in the spleen, lymph node, bone marrow, T-cells, and dendritic cells. Antibodies for BAFF can be obtained through a variety of commercial sources including, e.g., ABCAM®, ACRIS ANTIBODIES™, GENETEX™, LIFESPAN BIOSCIENCES™, SANTA CRUZ BIOTECHNOLOGY® and SIGMA-ALDRICH®.

IL-1RA (Also Known as IL-1RN):

The nucleotide and protein sequences of human IL-1RA are described in e.g., Carter, D B et al. (1990) Nature 344:633-638; Eisenberg, S P et al. (1990) Nature 343:341-346; Eisenberg, S P (1991) PNAS 88:5232-5236; Lennard, A. et al. (1992) Cytokine 4:83-89; Jenkins, J K et al. (1997) J Immunol 158:748-755; Haskill, S. et al. (1991) PNAS 88:3681-3685; Muzio, M et al. (1995) J Exp Med 182:623-628; Hannum, C H et al. (1990) Nature 343:336-340; and Nicklin, M J H et al. (2002) Genomics 79:718-725. IL-1RA is predominantly expressed in endothelial cells and is a member of the interleukin-1 cytokine family. IL-1RA functions to inhibit the activity of interleukin 1 alpha and interleukin 1 beta and modulates a variety of interleukin 1 related immune and inflammatory responses. Antibodies for IL-1RA can be purchased from a variety of commercial sources including, but not limited to, ABCAM®, ACRIS ANTIBODIES™, GENETEX™, NOVUS BIOLOGICALS®, and SANTA CRUZ BIOTECHNOLOGY®.

Interleukin-13 (IL-13):

The nucleotide and protein sequences of human IL-13 are disclosed in e.g., Minty, A J. et al. (1993) Nature 362: 248-250; McKenzie, A N et al. (1993) PNAS 90:3735-3739; Smirnov, D V et al. (1995) Gene 155:277-281; Dolganov, G et al. (1996) Blood 87:3316-3326; and Heinzmann, A. et al. (2000) Hum Mol Genet 9:549-559. IL-13 is an immunoregulatory cytokine produced primarily by activated Th2 cells and is involved in B-cell maturation and differentiation. IL-13 also down-regulates macrophage activity and inhibits production of pro-inflammatory cytokines and chemokines. IL-13 antibodies can be obtained from e.g., ABCAM®, ABD SEROTEC™, ABNOVA CORPORATION™, MILLIPORE™, R&D SYSTEMS®, THERMO SCIENTIFIC PIERCE ANTIBODIES™, ACRIS ANTIBODIES™, ANTIGENIX AMERICA™, and SANTA CRUZ BIOTECHNOLOGY®.

Monocyte Chemoattractant Protein-1 (MCP-1; Also Known as CCL2):

The nucleotide and protein sequences of human MCP-1 are described in e.g., Furutani, Y et al. (1989) Biochem Biophys Res Commun 159: 249-255; Rollins, B J et al. (1989) Mol Cell Biol 9:4687-4695; Yoshimura, T. et al. (1989) FEBS Lett 244:487-493; Chang, H C. et al. (1989) Int Immunol 1:388-397; Shyy, Y J et al. (1990) Biochem Biophys Res Commun 169:346-351; Li, Y S. et al. (1993) Mol Cell Biochem 126:61-68; and Finzer, P. et al. (2000) Oncogene 19:3235-3244. MCP-1 is structurally related to the CXC subfamily of cytokines and augments monocyte anti-tumor activity. MCP-1 displays chemotactic activity to recruit monocytes and basophils, but does not have chemotactic activity for neutrophils or eosinophils. Commercial antibodies for MCP-1 can be obtained from e.g., ABCAM®, MILLIPORE™, CELL SIGNALING TECHNOLOGY®, and NOVUS BIOLOGICALS®.

C-Reactive Protein (CRP):

The nucleotide and protein sequences of human CRP are described in e.g., Lei, K J et al. (1985) J Biol Chem 260:13377-13383; Woo, P. et al. (1985) J Biol Chem 260:13384-13388; Tucci, A. et al., (1983) J Immunol 131:2416-2419; Whitehead, A S. et al. (1983) Science 221:69-71; Oliveira, E B. et al. (1979) JBiol Chem 254:489-502; and Osmand, A P. et al. (1977) PNAS 74:1214-1218. CRP is a plasma protein that is induced by IL-1 and IL-6. Increased levels of CRP occur during acute phase response to tissue injury, infection or other inflammatory stimuli. Commercial antibodies for CRP can be obtained from e.g., MILLIPORE™, R&D SYSTEMS®, ABCAM®, and ADVANCED IMMUNOCHEMICAL INC™.

Beta-2-Microglobulin (B2M):

The nucleotide and protein sequences of human B2M are described in e.g., Guessow, D. et al. (1987) J Immunol 139:3132-3138; He, X H. et al. (2004) Sheng Wu Gong Cheng Xue Bao 20:99-103; Suggs, S V et al. (1981) PNAS 78:6613-6617; and Cunningham, B A et al. (1973) Biochemistry 12:4811-4822. B2M is associated with the major histocompatibility complex (MHC) class I heavy chain on the surface of nearly all nucleated cells. Commercial antibodies for B2M can be obtained from e.g., MILLIPORE™, ACRIS ANTIBODIES™, ABCAM®, PROTEIN TECH GROUP™ and SIGMA-ALDRICH®.

Ferritin:

The nucleotide and protein sequences for the human ferritin heavy chain and human ferritin light chain are disclosed in e.g., Constanzo F et al. (1984) EMBO J 3:23-27; Boyd, D. et al. (1985) JBiol Chem 260:11755-11761; Chou, C C et al. (1986) Nucleic Acids Research 14: 721-736; Hentze, M W et al. (1986) PNAS 83:7226-7230; Dhar, M. et al. (1993) Gene 126:275-278; Boyd, D. et al. (1984) PNAS 81:4751-4755; Dorner, M H et al. (1985) PNAS 82:3139-3143; Santoro, C. et al. (1986) 14: 2863-2876; and Addison, J et al. (1983) FEBS Lett 164:139-144. The human ferritin protein is made up of 24 subunits and comprises both ferritin heavy chain and ferritin light chain subunits. Human ferritin is found in nearly all cell types and plays a role in iron homeostasis and iron delivery to cells. Commercial antibodies for ferritin can be obtained from e.g., SANTA CRUZ BIOTECHNOLOGY®, THERMO SCIENTIFIC PIERCE ANTIBODIES™, COVALAB™, and SIGMA-ALDRICH®.

Tumor Necrosis Factor Receptor-2 (TNFR2; Also Known as TNFRII, TNFBR, TNFRSF1B):

The nucleotide and protein sequences of human TNFR2 are described in e.g., Kohno, T. et al. (1990) PNAS 87:8331-8335; Smith, C A et al. (1990) Science 248:1019-1023; Beltinger, C P et al. (1996) Genomics 35:94-100; Lainez, B. et al. (2004) Int Immunol 16:169-177; Loetscher, H. et al. (1990) J Biol Chem 265:20131-20138; Dembic, Z. et al. (1990) Cytokine 2:231-237; and Pennica, D M et al. (1992) JBiol Chem 267:21172-21178. TNFR2 is a member of the TNF-receptor superfamily and forms a hetercomplex with TNF-receptor 1 to recruit two anti-apoptotic proteins, c-IAP1 and c-IAP2. Thus, TNFR2 is thought to block TNF-alpha-induced apoptosis and regulate TNF-alpha function by antagonizing its biological activity. Commercial antibodies for TNFR2 can be obtained from e.g., ACRIS ANTIBODIES™, ABCAM®, PROTEIN TECH GROUP™, LIFESPAN BIOSCIENCES™, GENETEX™, and CELL SIGNALING TECHNOLOGY®.

The protein levels of the biomarkers identified in Table 1 and Table 2 can be used alone or in combination (i.e., two or more) to assess the likelihood of a subject to respond to interferon-β therapy. In some embodiments, two or more of the biomarkers in Table 1 and Table 2 (e.g., 3, 4, 5, 6, 7, 8, or 9 (i.e., all)) are used in combination to assess responsiveness of a subject to interferon-β. In one embodiment, CCL2 is used as a biomarker with the methods described herein. In another embodiment, CCL2 and BAFF are used as a biomarker using the methods described herein. In another embodiment, CCL2, BAFF and at least one additional biomarker (e.g., 1, 2, 4, 5, 6, or 7) from Table 1 and Table 2 are used as a panel of biomarkers using the methods described herein. The methods provided herein are particularly useful for identifying subjects that are likely to respond to IFNβ treatment (e.g. IFNβ-1A, IFNβ-1B, or a derivative thereof (e.g., a pegylated derivative)) prior to initiation of such treatment (e.g., pre-therapy) or early in the therapeutic regimen. In some embodiments, expression of one or more biomarkers from Table 1 and Table 2 are measured in a subject at least 2 weeks, at least 1 month, at least 3 months, at least 6 months, or at least 1 year after initiation of therapy. In some embodiments, it is preferred that expression of one or more biomarkers of Table 1 and Table 2 are measured less than 6 months after initiation of therapy to permit the skilled practitioner to switch the subject to a different therapeutic strategy. Thus, in some embodiments it is preferred that expression of one or more biomarkers of Table 1 and Table 2 are measured within 1-6 months, 1-5 months, 1-4 months, 1-3 months, 1-2 months, 2-6 months, 3-6 months, 4-6 months, 5-6 months, 2-3 months, 3-4 months, or 4-5 months of initiation of IFNβ-1A therapy. In some embodiments, the expression of one or more biomarkers is determined 3-6 months after initiation of therapy (e.g., 3 months, 3.5 months, 4 months, 4.5 months, 5 months, 5.5 months, 6 months).

The methods described herein can also be used to monitor a positive response of a subject to treatment with IFNβ. Such methods are useful for early detection of tolerance to IFNβ therapy or to predict whether a subject will shift from a responder to a non-responder phenotype. In such embodiments, the level (e.g., expression) of one or more of the biomarkers in Table 1 and Table 2 are determined e.g., at least every 2 weeks, at least every month, at least every 2 months, at least every 3 months, at least every 4 months, at least every 5 months, at least every 6 months, at least every 7 months, at least every 8 months, at least every 9 months, at least every 10 months, at least every 11 months, at least every year, at least every 18 months, at least every 2 years, at least every 3 years, at least every 5 years or more. It is also contemplated that expression of the biomarkers is at irregular intervals e.g., biomarkers can be detected in an individual at 3 months of treatment, at 6 months of treatment, and at 7 months of treatment. Thus, in some embodiments, the expression of the biomarkers is determined when deemed necessary by the skilled physician monitoring treatment of the subject.

The methods described herein can be used in any subject having multiple sclerosis including sub-types such as benign MS, quiescent relapsing-remitting MS, active relapsing-remitting MS, primary progressive MS, and secondary progressive MS. It is also contemplated, in other embodiments, that the methods can be used in subjects having MS-like symptoms, such as those having clinically isolated syndrome (CIS) or clinically defined MS (CDMS). Clinically isolated syndrome (CIS) refers to the detection of a single clinical episode of demyelination or other monophasic CNS inflammatory disorder (e.g., Spinal Cord Syndrome, Brainstem/Cerebellar Syndrome, and others described below). Frohman et al. (2003) Neurology 2003 61(5):602-11 report that, in subjects with CIS, three or more white matter lesions on a T2-weighted MRI scan (especially if one of these lesions is located in the periventricular region) is a very sensitive predictor (>80%) of the subsequent development of CDMS within the next 7 to 10 years. In a preferred embodiment, the methods described herein are used to assess expression of one or more biomarkers of Table 1 in a subject having RRMS.

A subject that is identified as a responder using the methods described herein can be treated with any IFNβ agent known in the art presently or to be developed (e.g. IFNβ-1A, IFNβ-1B, or a derivative thereof (e.g., a pegylated derivative)). In one embodiment, the IFNβ agent is an IFNβ-1A agent (e.g., AVONEX®, REBIF®). In another embodiment, the IFNβ agent is an IFNβ-1B agent (e.g., BETASERON®, BETAFERON®).

In some embodiments, the amount of the biomarker determined in a serum sample from a subject is quantified as an absolute measurement (e.g., ng/mL). Absolute measurements can easily be compared to a reference value or cut-off value. For example, a cut-off value can be determined that represents a non-responder status; any absolute values falling either above (i.e., for biomarkers that increase expression with MS) or falling below (i.e., for biomarkers with decreased expression in MS) the cut-off value are likely to be non-responders to IFNβ therapy.

Alternatively, the relative amount of a biomarker is determined. In one embodiment, the relative amount is determined by comparing the expression of one or more serum biomarkers in a subject with MS to the expression of the serum biomarkers in a healthy control subject. In another embodiment, the relative amount is determined by comparing the expression of one or more serum biomarkers in a subject with MS at two or more timepoints (e.g., at baseline and 3 months after initiation of therapy or 3 and 6 months after initiation of therapy).

The present invention also pertains to the field of predictive medicine in which diagnostic assays, pharmacogenomics, and monitoring clinical trials are used for predictive purposes to thereby treat an individual prophylactically. Accordingly, one aspect of the present invention relates to assays for determining the amount, structure, and/or activity of polypeptides or nucleic acids corresponding to one or more markers of the invention, in order to determine whether an individual having multiple sclerosis or at risk of developing multiple sclerosis will be more likely to respond to IFN-β-mediated therapy.

Accordingly, in one aspect, the invention is drawn to a method for determining whether a subject with multiple sclerosis is likely to respond to treatment with an IFN-β agent. In another aspect, the invention is drawn to a method for predicting a time course of disease. In still another aspect, the method is drawn to a method for predicting a probability of a significant event in the time course of the disease (e.g., relapse or shift from responder to non-responder status). In certain embodiments, the method comprises detecting a biomarker or combination of biomarkers associated with responsiveness to treatment with an IFN-β agent as described herein and determining whether the subject is likely to respond to treatment with the IFN-β agent (e.g. IFNβ-1A, IFNβ-1B, or a derivative thereof (e.g., a pegylated derivative)).

In some embodiments, the methods involve evaluation of a biological sample e.g., a serum sample from a subject, e.g., a patient who has been diagnosed with or is suspected of having multiple sclerosis (e.g., presents with symptoms of multiple sclerosis) to detect changes in one or more biomarkers described herein (e.g., gene expression or polypeptide levels).

The results of the screening method and the interpretation thereof are predictive of the patient's response to treatment with IFN-β agents (e.g., AVONEX® (interferon beta 1a), REBIF® (interferon beta 1a), BETASERON® (interferon beta 1b), BETAFERON® (interferon beta 1b)), alone or in combination with symptom management agents. According to the present invention, alterations in expression of one or more biomarkers described herein, e.g., CCL21, BAFF, IL-1RA, IL-13, MCP-1, CRP, B2M, ferritin, and TNFR2 is indicative that treatment with IFN-β agents will provide enhanced therapeutic benefit for patients with multiple sclerosis relative to healthy controls.

In yet another embodiment, the one or more alterations, e.g., alterations in biomarker expression are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to worsening disease. In another embodiment, the significant event is the progression from primary diagnosis to relapse. In another embodiment, the significant event is the progression from secondary MS to death. In another embodiment, the significant event is the progression from remission to relapse. In another embodiment, the significant event is the progression from relapse to death. In certain embodiments, the time course is measured with respect to one or more overall survival rate, time to progression and/or using the EDSS or other assessment criteria.

### Methods for Detection or Determining MS Biomarkers

### Polypeptide Detection

Methods to measure biomarkers of this invention, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners.

The activity or level of a marker protein can also be detected and/or quantified by detecting or quantifying the expressed polypeptide. The polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

Another agent for detecting a polypeptide of the invention is an antibody capable of binding to a polypeptide corresponding to a marker of the invention, e.g., an antibody with a detectable label. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Immunohistochemistry or IHC refers to the process of localizing antigens (e.g. proteins) in cells of a tissue section exploiting the principle of antibodies binding specifically to antigens in biological tissues. Specific molecular markers are characteristic of particular cellular events such as proliferation or cell death (apoptosis). IHC is also widely used in research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore, such as fluorescein, rhodamine, DyLight Fluor or Alexa Fluor.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, New York).

In one format, antibodies, or antibody fragments, can be used in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies can be directly labeled or alternatively can be subsequently detected using labeled antibodies (e.g., labeled sheep anti-human antibodies) that specifically bind to the anti-polypeptide.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

In another embodiment, the polypeptide is detected and/or quantified using LUMINEX™ assay technology. The LUMINEX™ assay separates tiny color-coded beads into e.g., distinct sets that are each coated with a reagent for a particular bioassay, allowing the capture and detection of specific analytes from a sample in a multiplex manner. The LUMINEX™ assay technology can be compared to a multiplex ELISA assay using bead-based fluorescence cytometry to detect analytes such as biomarkers.

Immunological binding assays (or immunoassays) typically utilize a “capture agent” to specifically bind to and often immobilize the analyte (polypeptide or subsequence). The capture agent is a moiety that specifically binds to the analyte. In another embodiment, the capture agent is an antibody that specifically binds a polypeptide. The antibody (anti-peptide) can be produced by any of a number of means well known to those of skill in the art.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one embodiment, the labeling agent is a second human antibody bearing a label. Alternatively, the second antibody can lack a label, but it can, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Exemplary immunoassays for detecting a polypeptide can be competitive or noncompetitive. Noncompetitive immunoassays are assays in which the amount of captured analyte is directly measured. In one “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In another embodiment, the antibody is immobilized on a solid substrate. The amount of polypeptide bound to the antibody can be determined either by measuring the amount of polypeptide present in a polypeptide/antibody complex, or alternatively by measuring the amount of remaining uncomplexed polypeptide. The amount of polypeptide can be detected by providing a labeled polypeptide.

The assays described herein are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described herein.

In another embodiment, level (activity) is assayed by measuring the enzymatic activity of the gene product. Methods of assaying the activity of an enzyme are well known to those of skill in the art.

In vivo techniques for detection of a marker protein include introducing into a subject a labeled antibody directed against the protein. For example, the antibody can be labeled with a radioactive marker whose presence and location in a subject can be detected by standard imaging techniques.

Certain markers identified by the methods of the invention can be secreted proteins. It is a simple matter for the skilled artisan to determine whether any particular marker protein is a secreted protein. In order to make this determination, the marker protein is expressed in, for example, a mammalian cell, e.g., a human cell line, extracellular fluid is collected, and the presence or absence of the protein in the extracellular fluid is assessed (e.g., using a labeled antibody which binds specifically with the protein).

The following is an example of a method which can be used to detect secretion of a protein. About 8×10⁵ 293 T cells are incubated at 37° C. in wells containing growth medium (Dulbecco's modified Eagle's medium {DMEM} supplemented with 10% fetal bovine serum) under a 5% (v/v) CO2, 95% air atmosphere to about 60-70% confluence. The cells are then transfected using a standard transfection mixture comprising 2 micrograms of DNA comprising an expression vector encoding the protein and 10 microliters of LIPOFECTAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

The invention also encompasses kits for detecting the presence of a polypeptide or nucleic acid corresponding to a marker of the invention in a biological sample, e.g., a sample containing tissue, whole blood, serum, plasma, buccal scrape, saliva, cerebrospinal fluid, urine, stool, and bone marrow. Such kits can be used to determine if a subject is suffering from or is at increased risk of developing multiple sclerosis. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

### Proteins and Antibody Detection

One aspect of the invention pertains to isolated proteins which correspond to one or more markers of the invention, and biologically active portions thereof. In one embodiment, the native polypeptide corresponding to a marker can be isolated from a biological sample (e.g., a blood sample, a serum sample, a non-cell sample, a cell sample or a tissue sample) by an appropriate purification scheme using standard protein purification techniques. In a preferred embodiment, the proteins are isolated from a serum sample. In another embodiment, the proteins are isolated from peripheral blood mononuclear cells. In another embodiment, the proteins are isolated from a cell-free sample.

In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the biological sample, cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, less than about 20%, less than about 10%, or less than about 5% (by dry weight) of heterologous protein (also referred to herein as a “contaminating protein”). When the protein or biologically active portion thereof is recombinantly produced, it can be substantially free of culture medium, i.e., culture medium represents less than about 20%, less than about 10%, or less than about 5% of the volume of the protein preparation. When the protein is produced by chemical synthesis, it can substantially be free of chemical precursors or other chemicals, i.e., it is separated from chemical precursors or other chemicals which are involved in the synthesis of the protein. Accordingly such preparations of the protein have less than about 30%, less than about 20%, less than about 10%, less than about 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the gene products described herein, e.g., CCL21, BAFF, IL-1RA, IL-13, MCP-1, CRP, B2M, ferritin, and TNFR2 identified herein of the present invention, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

In certain embodiments, the polypeptide has an amino acid sequence of a protein encoded by a nucleic acid molecule disclosed herein. Other useful proteins are substantially identical (e.g., at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 99.5% or greater) to one of these sequences and retain the functional activity of the protein of the corresponding full-length protein yet differ in amino acid sequence.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. Another, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (or at least 10, at least 15, at least 20, or at least 30 or more) amino acid residues of the amino acid sequence of one of the polypeptides of the invention, and encompasses an epitope of the protein such that an antibody raised against the peptide forms a specific immune complex with a marker of the invention to which the protein corresponds. Exemplary epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to, for example, determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

### Methods for Detection of Gene Expression

Marker expression level can also be assayed. Expression of a marker of the invention can be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see e.g., Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that can contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

In another embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Exemplary nucleic acid probes are 20 bases or longer in length (See, e.g., Sambrook et al. for methods of selecting nucleic acid probe sequences for use in nucleic acid hybridization). Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a healthy subject, or between samples from different sources.

Alternatively, the expression level can be provided as a relative expression level. To determine a relative expression level of a marker, the level of expression of the marker is determined for 10 or more samples of normal versus MS isolates, or even 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In certain embodiments, the samples used in the baseline determination will be from samples derived from a subject having multiple sclerosis versus samples from a healthy subject of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the multiple sclerosis disease state.

In another embodiment, expression of a marker is assessed by preparing genomic DNA or mRNA/cDNA (i.e., a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g., at least 7, at least 10, at least 15, at least 20, at least 25, at least 30, at least 40, at least 50, at least 100, at least 500, or more nucleotide residues) of a marker of the invention. If polynucleotides complementary to or homologous with a marker of the invention are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, the hybridization can be performed under stringent hybridization conditions.

In another embodiment, a combination of methods to assess the expression of a marker is utilized.

Because the compositions, kits, and methods of the invention rely on detection of a difference in expression levels of one or more markers of the invention, in certain embodiments the level of expression of the marker is significantly greater than the minimum detection limit of the method used to assess expression in at least one of a biological sample from a subject with MS or a healthy control.

### Nucleic Acid Molecules and Probes

One aspect of the invention pertains to isolated nucleic acid molecules that correspond to one or markers of the invention, including nucleic acids which encode a polypeptide corresponding to one or more markers of the invention or a portion of such a polypeptide. The nucleic acid molecules of the invention include those nucleic acid molecules which reside in genomic regions identified herein. Isolated nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In one embodiment, the nucleic acids are isolated from a e.g., blood sample or peripheral blood mononuclear cells (PBMCs).

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

If so desired, a nucleic acid molecule of the present invention, e.g., the marker gene products identified herein (e.g., the markers set forth in Table 1), can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N. Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, at least about 15, at least about 25, at least about 50, at least about 75, at least about 100, at least about 125, at least about 150, at least about 175, at least about 200, at least about 250, at least about 300, at least about 350, at least about 400, at least about 500, at least about 600, at least about 700, at least about 800, at least about 900, at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 15 kb, at least about 20 kb, at least about 25 kb, at least about 30 kb, at least about 35 kb, at least about 40 kb, at least about 45 kb, at least about 50 kb, at least about 60 kb, at least about 70 kb, at least about 80 kb, at least about 90 kb, at least about 100 kb, at least about 200 kb, at least about 300 kb, at least about 400 kb, at least about 500 kb, at least about 600 kb, at least about 700 kb, at least about 800 kb, at least about 900 kb, at least about 1 mb, at least about 2 mb, at least about 3 mb, at least about 4 mb, at least about 5 mb, at least about 6 mb, at least about 7 mb, at least about 8 mb, at least about 9 mb, at least about 10 mb or more consecutive nucleotides of a nucleic acid of the invention.

Probes based on the sequence of a nucleic acid molecule of the invention can be used to detect transcripts (e.g., mRNA) or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that are substantially homologous to the gene products described herein, e.g., IFN-β signaling pathway gene products identified herein (e.g., the markers set forth in Table 1) such that they are at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater. In other embodiments, the invention further encompasses nucleic acid molecules that are substantially homologous to the gene products described herein, e.g., IFN-β pathway gene products identified herein (e.g., the markers set forth in Table 1) such that they differ by only or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1 kb, at least 2 kb, at least 3 kb, at least 4 kb, at least 5 kb, at least 6 kb, at least 7 kb, at least 8 kb, at least 9 kb, at least 10 kb, at least 15 kb, at least 20 kb, at least 25 kb, at least 30 kb, at least 35 kb, at least 40 kb, at least 45 kb, at least 50 kb nucleotides or any range in between.

In another embodiment, an isolated nucleic acid molecule of the invention is at least 7, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 550, at least 650, at least 700, at least 800, at least 900, at least 1000, at least 1200, at least 1400, at least 1600, at least 1800, at least 2000, at least 2200, at least 2400, at least 2600, at least 2800, at least 3000, at least 3500, at least 4000, at least 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60%, at least 65%, at least 70%, at least 75%, at least 80%, or at least 85% identical to each other typically remain hybridized to each other. Such stringent conditions are known to those skilled in the art and can be found in e.g., sections 6.3.1-6.3.6 of Current Protocols in Molecular Biology, John Wiley & Sons, N.Y. (1989). Another, non-limiting example of stringent hybridization conditions are hybridization in 6× sodium chloride/sodium citrate (SSC) at about 45° C., followed by one or more washes in 0.2×SSC, 0.1% SDS at 50-65° C.

The methods described herein can also include molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### Kits

A kit is any manufacture (e.g., a package or container) comprising at least one reagent, e.g., a probe or an antibody, for specifically detecting a marker of the invention, the manufacture being promoted, distributed, or sold as a unit for performing the methods of the present invention. When the compositions, kits, and methods of the invention are used for carrying out the methods of the invention, probes/antibodies corresponding to one or more of the markers set forth in Table 1 can be selected such that a positive result is obtained in at least about 20%, at least about 40%, at least about 60%, at least about 80%, at least about 90%, at least about 95%, at least about 99% or in 100% of subjects afflicted with multiple sclerosis, of the corresponding sub-type, or relapsing/remitting nature. In certain embodiments, the marker or panel of markers of the invention can be selected such that a PPV (positive predictive value) of greater than about 10% is obtained for the general population (e.g., coupled with an assay specificity greater than 99.5%).

When a plurality of biomarkers described herein are measured, e.g., probes/antibodies for the markers set forth in Table 1 are used in the compositions, kits, and methods of the invention, the amount, structure, and/or activity of each marker or level of expression or copy number can be compared with the normal amount, structure, and/or activity of each of the plurality of markers or level of expression in samples of the same type obtained from a subject having multiple sclerosis, either in a single reaction mixture (i.e., using reagents, such as different fluorescent probes, for each marker) or in individual reaction mixtures corresponding to one or more of the biomarkers described herein, e.g., gene products identified herein (e.g., the markers set forth in Table 1). If a plurality of gene products (e.g., the markers set forth in Table 1 or described herein) is used, then 1, 2, 3, 4, 5, 6, 7, 8, 9, or more individual markers can be used or identified.

The invention includes compositions, kits, and methods for assaying serum in a sample (e.g., a sample obtained from a subject). These compositions, kits, and methods are substantially the same as those described above, except that, where necessary, the compositions, kits, and methods are adapted for use with certain types of samples. For example, when the sample is a serum sample, it can be necessary to adjust the ratio of compounds in the compositions of the invention, in the kits of the invention, or the methods used. Such methods are well known in the art and within the skill of the ordinary artisan.

The invention thus includes a kit for assessing the responsiveness of a subject having multiple sclerosis to treatment using an IFN-β agent (e.g., in a sample such as a serum sample). The kit can comprise one or more reagents capable of identifying one or more of the markers set forth in Table 1, e.g., binding specifically with a nucleic acid or polypeptide corresponding one or more of the biomarkers described herein, e.g., gene products identified herein (e.g., the markers set forth in Table 1). Suitable reagents for binding with a polypeptide corresponding to a marker of the invention include antibodies, antibody derivatives, antibody fragments, and the like. Suitable reagents for binding with a nucleic acid (e.g., a genomic DNA, an mRNA, a spliced mRNA, a cDNA, or the like) include complementary nucleic acids. For example, the nucleic acid reagents can include oligonucleotides (labeled or non-labeled) fixed to a substrate, labeled oligonucleotides not bound with a substrate, pairs of PCR primers, molecular beacon probes, and the like.

The kit of the invention can optionally comprise additional components useful for performing the methods of the invention. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a reference sample for comparison of expression levels of the biomarkers described herein, and the like.

A kit of the invention can comprise a reagent useful for determining protein level or protein activity of a marker.

### MS Therapeutic Agents, Compositions and Administration

There are several medications presently used to modify the course of multiple sclerosis in patients. Such agents include, but are not limited to, Beta interferons (e.g., AVONEX® (interferon beta 1a), REBIF® (interferon beta 1a), BETASERON® (interferon beta 1b), BETAFERON® (interferon beta 1b), among others)), glatiramer (COPAXONE®), natalizumab (TYSABRI®), and mitoxantrone (NOVANTRONE®).

### IFN-β Agents (Beta Interferons)

One known therapy for MS includes treatment with interferon beta. Interferons (IFNs) are natural proteins produced by the cells of the immune systems of most animals in response to challenges by foreign agents such as viruses, bacteria, parasites and tumor cells. Interferons belong to the large class of glycoproteins known as cytokines. Interferon beta has 165 amino acids. Interferons alpha and beta are produced by many cell types, including T-cells and B-cells, macrophages, fibroblasts, endothelial cells, osteoblasts and others, and stimulate both macrophages and NK cells. Interferon gamma is involved in the regulation of immune and inflammatory responses. It is produced by activated T-cells and Th1 cells.

Several different types of interferon are now approved for use in humans. Interferon alpha (including forms interferon alpha-2a, interferon alpha-2b, and interferon alfacon-1) was approved by the United States Food and Drug Administration (FDA) as a treatment for Hepatitis C. There are two currently FDA-approved types of interferon beta. Interferon beta 1a (AVONEX®) is identical to interferon beta found naturally in humans, and interferon beta 1b (BETASERON®) differs in certain ways from interferon beta 1a found naturally in humans, including that it contains a serine residue in place of a cysteine residue at position 17. Other uses of interferon beta have included treatment of AIDS, cutaneous T-cell lymphoma, Acute Hepatitis C (non-A, non-B), Kaposi's sarcoma, malignant melanoma, and metastatic renal cell carcinoma.

IFN-β agents can be administered to the subject by any method known in the art, including systemically (e.g., orally, parenterally, subcutaneously, intravenously, rectally, intramuscularly, intraperitoneally, intranasally, transdermally, or by inhalation or intracavitary installation). Typically, the IFN-β agents are administered subcutaneously, or intramuscularly.

IFN-β agents can be used to treat those subjects determined to be “responders” using the methods described herein. In one embodiment, the IFN-β agents are used as a monotherapy (i.e., as a single “disease modifying therapy”) although the treatment regimen can further comprise the use of “symptom management therapies” such as antidepressants, analgesics, anti-tremor agents, etc. In one embodiment, the IFN-β agent is an IFNβ-1A agent (e.g., AVONEX®, REBIF®). In another embodiment, the INF-β agent is an INFβ-1B agent (e.g., BETASERON®, BETAFERON®).

AVONEX®, an Interferon 13-1a, is indicated for the treatment of patients with relapsing forms of MS that are determined to be responders using the methods described herein to slow the accumulation of physical disability and decrease the frequency of clinical exacerbations. AVONEX® (Interferon beta-1a) is a 166 amino acid glycoprotein with a predicted molecular weight of approximately 22,500 daltons. It is produced by recombinant DNA technology using genetically engineered Chinese Hamster Ovary cells into which the human interferon beta gene has been introduced. The amino acid sequence of AVONEX® (interferon beta 1a) is identical to that of natural human interferon beta. The recommended dosage of AVONEX® (Interferon beta-1a) is 30 mcg injected intramuscularly once a week. AVONEX® (interferon beta 1a) is commercially available as a 30 mcg lyophilized powder vial or as a 30 mcg prefilled syringe.

Interferon beta Ia (AVONEX®) is identical to interferon beta found naturally in humans (AVONEX®, i.e., Interferon beta Ia (SwissProt Accession No. P01574 and gi:50593016). The sequence of interferon beta is:

(SEQ ID NO: 1)
MTNKCLLQIALLLCFSTTALSMSYNLLGFLQRSSNFQCQKLLWQLNGRL
EYCLKDRMNFDIPEEIKQLQQFQKEDAALTIYEMLQNIFAIFRQDSSST
GWNETIVENLLANVYHQINHLKTVLEEKLEKEDFTRGKLMSSLHLKRYY
GRILHYLKAKEYSHCAWTIVRVEILRNFYFINRLTGYLRN.

Methods for making AVONEX® (interferon beta 1a) are known in the art.

Treatment of responders identified using the methods described herein further contemplates that compositions (e.g., IFN beta 1a molecules) having biological activity that is substantially similar to that of AVONEX® (interferon beta 1a) will permit successful treatment similar to treatment with AVONEX® (interferon beta 1a) when administered in a similar manner. Such other compositions include, e.g., other interferons and fragments, analogues, homologues, derivatives, and natural variants thereof with substantially similar biological activity. In one embodiment, the INF-β agent is modified to increase one or more pharmacokinetic properties. For example, the INF-β agent can be a modified form of interferon 1a to include a pegylated moiety. PEGylated forms of interferon beta 1a are described in, e.g., Baker, D. P. et al. (2006) Bioconjug Chem 17(1):179-88; Arduini, R M et al. (2004) Protein Expr Purif 34(2):229-42; Pepinsky, R B et al. (2001) J. Pharmacol. Exp. Ther. 297(3):1059-66; Baker, D. P. et al. (2010) J Interferon Cytokine Res 30(10):777-85 (all of which are incorporated herein by reference in their entirety, and describe a human interferon beta 1a modified at its N-terminal alpha amino acid to include a PEG moiety, e.g., a 20 kDa mPEG-O-2-methylpropionaldehyde moiety). Pegylated forms of IFN beta 1a can be administered by, e.g., injectable routes of administration (e.g., subcutaneously).

REBIF® is also an Interferon β-1a agent, while BETASERON® and BETAFERON® are Interferon β 1b agents. Both REBIF® (interferon beta 1a) and BETASERON® (interferon beta 1b) are formulated for administration by subcutaneous injection.

Dosages of IFN-β agents to administer can be determined by one of skill in the art, and include clinically acceptable amounts to administer based on the specific interferon-beta agent used. For example, AVONEX® (interferon beta 1a) is typically administered at 30 microgram once a week via intramuscular injection. Other forms of interferon beta 1a, specifically REBIF® (interferon beta 1a), is administered, for example, at 22 microgram three times a week or 44 micrograms once a week, via subcutaneous injection. Interferon beta-1A can be administered, e.g., intramuscularly, in an amount of between 10 and 50 μg. For example, AVONEX® (interferon beta 1a) can be administered every five to ten days, e.g., once a week, while REBIF® (interferon beta 1a) can be administered three times a week.

### Non-IFN-β Agents

In other embodiments, alternative therapies to the IFN-β agent can be administered. For example, in subjects determined to be non-responders using the methods described herein, a skilled physician can select a therapy that includes a non-IFN-β agent that can act as a “disease modifying therapy” e.g., glatiramer (COPAXONE®), natalizumab (TYSABRI®, ANTEGREN®), and mitoxantrone (NOVANTRONE®).

In one embodiment, the alternative therapy includes a polymer of four amino acids found in myelin basic protein, e.g., a polymer of glutamic acid, lysine, alanine and tyrosine (e.g., glatiramer (COPAXONE®)). In other embodiments, the alternative therapy includes an antibody or fragment thereof against alpha-4 integrin (e.g., natalizumab (TYSABRI®)). In yet other embodiments, the alternative therapy includes an anthracenedione molecule (e.g., mitoxantrone (NOVANTRONE®)). In yet another embodiment, the alternative therapy includes a fingolimod (e.g., FTY720; GILENYA®). In one embodiment, the alternative therapy is a dimethyl fumarate (e.g., an oral dimethyl fumarate (BG-12)). In other embodiments, the alternative therapy is an antibody to the alpha subunit of the IL-2 receptor of T cells (e.g., Daclizumab; described in, e.g., Rose, J. W. et al. (2007) Neurology 69 (8): 785-789). In yet other embodiments, the alternative therapy is an antibody against CD52 (e.g., alemtuzumab (LEMTRADA®)). In yet another embodiment, the alternative therapy includes an anti-LINGO-1 antibody (described in, e.g., U.S. Pat. No. 8,058,406, entitled “Composition comprising antibodies to LINGO or fragments thereof.”).

Steroids, e.g., corticosteroid, and ACTH agents can be used to treat acute relapses in relapsing-remitting MS or secondary progressive MS. Such agents include, but are not limited to, DEPO-MEDROL® (methylprednisolone acetate), SOLU-MEDROL® (methylprednisolone sodium succinate), DELTASONE® (prednisone), DELTA-CORTEF® (prednisolone), MEDROL® (methylprednisolone), DECADRON® (dexamethasone), and ACTHAR® (corticotropin).

Doses and modes of administration of the non-IFNβ agent are known in the art.

### Symptom Management

In certain embodiments, the method further includes the use of one or more symptom management therapies, such as antidepressants, analgesics, anti-tremor agents, among others. Treatment of a subject with a disease modifying IFN-β agent or non-IFN-β agent can be combined with one or more of the following therapies often used in symptom management of subjects having MS: IMURAN® (azathioprine), CYTOXAN® (cyclophosphamide), NEOSAR® (cyclophosphamide), SANDIMMUNE® (cyclosporine), methotrexate, LEUSTATIN® (cladribine), TEGRETOL® (carbamazepine), EPITOL® (carbamazepine), ATRETOL® (carbamazepine), CARBATROL®-(carbamazepine), NEURONTIN® (gabapentin), TOPAMAX® (topiramate), ZONEGRAN® (zonisamide), DILANTIN® (phenytoin), NORPRAMIN® (desipramine), ELAVIL® (amitriptyline), TOFRANIL® (imipramine), IMAVATE® (imipramine), JANIMINE® (imipramine), SINEQUAN® (doxepine), ADAPIN® (doxepine), TRIADAPIN® (doxepine), ZONALON® (doxepine), VIVACTIL® (protriptyline), MARINOL® (synthetic cannabinoids), TRENTAL® (pentoxifylline), NEUROFEN® (ibuprofen), aspirin, acetaminophen, ATARAX® (hydroxyzine), PROZAC® (fluoxetine), ZOLOFT® (sertraline), LUSTRAL® (sertraline), EFFEXOR XR® (venlafaxine), CELEXA® (citalopram), PAXIL® (paroxetine), SEROXAT® (paroxetine), DESYREL® (trazodone), TRIALODINE® (trazodone), PAMELOR® (nortriptyline), AVENTYL® (imipramine), PROTHIADEN® (dothiepin), GAMANIL® (lofepramine), PARNATE® (tranylcypromine), MANERIX® (moclobemide), AURORIX® (moclobemide), WELLBUTRIN SR® (bupropion), AMFEBUTAMONE® (bupropion), SERZONE® (nefazodone), REMERON® (mirtazapine), AMBIEN® (zolpidem), XANAX® (alprazolam), RESTORIL® (temazepam), VALIUM® (diazepam), BUSPAR® (buspirone), SYMMETREL® (amantadine), CYLERT® (pemoline), PROVIGIL® (modafinil), DITROPAN XL® (oxybutynin), DDAVP® (desmopressin, vasopressin), DETROL® (tolterodine), URECHOLINE® (bethane), DIBENZYLINE® (phenoxybenzamine), HYTRIN® (terazo sin), PRO-BANTHINE® (propantheline), URISPAS® (hyoscyamine), CYSTOPAS® (hyoscyamine), LIORESAL® (baclofen), HIPREX® (methenamine), MANDELAMINE® (metheneamine), MACRODANTIN® (nitrofurantoin), PYRIDIUM® (phenazopyridine), CIPRO® (ciprofloxacin), DULCOLAX® (bisacodyl), BISACOLAX® (bisacodyl), SANI-SUPP® (glycerin), METAMUCIL® (psyllium hydrophilic mucilloid), FLEET ENEMA® (sodium phosphate), COLACE® (docusate), THEREVAC PLUS® (benzocaine), KLONOPIN® (clonazepam), RIVOTRIL® (clonazepam), DANTRIUM® (dantrolen sodium), CATAPRES® (clonidine), BOTOX® (botulinum toxin), NEUROBLOC® (botulinum toxin), ZANAFLEX® (tizanidine), SIRDALUD® (tizanidine), MYSOLINE® (primidone), DIAMOX® (acetozolamide), SINEMET® (levodopa, carbidopa), LANIAZID® (isoniazid), NYDRAZID® (isoniazid), ANTIVERT® (meclizine), BONAMINE® (meclizine), DRAMAMINE® (dimenhydrinate), COMPAZINE® (prochlorperazine), TRANSDERM® (scopolamine), BENADRYL® (diphenhydramine), ANTEGREN® (natalizumab), CAMPATH-1H® (alemtuzumab), FAMPRIDINE® (4-aminopyridine), GAMMAGARD® (IV immunoglobulin), GAMMAR-IV® (IV immunoglobulin), GAMIMUNE N® (IV immunoglobulin), IVEEGAM® (IV immunoglobulin), PANGLOBULIN® (IV immunoglobulin), SANDOGLOBULIN® (IV immunoglobulin), VENOBLOGULIN® (IV immunoglobulin), pregabalin, ziconotide, and AnergiX-MS®(MHC class II complexed with MS peptide).

It is also contemplated herein that a subject identified as a non-responder will be treated with one or more agents described herein to manage symptoms.

### Therapeutic Methods

“Treat,” “treatment,” and other forms of this word refer to the administration of an IFN-β agent, alone or in combination with one or more symptom management agents, to a subject, e.g., an MS patient, to impede progression of multiple sclerosis, to induce remission, to extend the expected survival time of the subject and or reduce the need for medical interventions (e.g., hospitalizations). In those subjects, treatment can include, but is not limited to, inhibiting or reducing one or more symptoms such as numbness, tingling, muscle weakness; reducing relapse rate, reducing size or number of sclerotic lesions; inhibiting or retarding the development of new lesions; prolonging survival, or prolonging progression-free survival, and/or enhanced quality of life.

As used herein, unless otherwise specified, the terms “prevent,” “preventing” and “prevention” contemplate an action that occurs before a subject begins to suffer from the a multiple sclerosis relapse and/or which inhibits or reduces the severity of the disease.

As used herein, and unless otherwise specified, the terms “manage,” “managing” and “management” encompass preventing the progression of MS symptoms in a patient who has already suffered from the disease, and/or lengthening the time that a patient who has suffered from MS remains in remission. The terms encompass modulating the threshold, development and/or duration of MS, or changing the way that a patient responds to the disease.

As used herein, and unless otherwise specified, a “therapeutically effective amount” of a compound is an amount sufficient to provide a therapeutic benefit in the treatment or management of multiple sclerosis, or to delay or minimize one or more symptoms associated with MS. A therapeutically effective amount of a compound means an amount of therapeutic agent, alone or in combination with other therapeutic agents, which provides a therapeutic benefit in the treatment or management of MS. The term “therapeutically effective amount” can encompass an amount that improves overall therapy, reduces or avoids symptoms or causes of the disease, or enhances the therapeutic efficacy of another therapeutic agent.

As used herein, and unless otherwise specified, a “prophylactically effective amount” of a compound is an amount sufficient to prevent relapse of MS, or one or more symptoms associated with the disease, or prevent its recurrence. A prophylactically effective amount of a compound means an amount of the compound, alone or in combination with other therapeutic agents, which provides a prophylactic benefit in the prevention of MS relapse. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug.

The methods described herein permit one of skill in the art to identify a monotherapy that an MS patient is most likely to respond to, thus eliminating the need for administration of multiple therapies to the patient to ensure that a therapeutic effect is observed. However, in one embodiment, combination treatment of an individual with MS is contemplated.

It will be appreciated that the IFN-β agent, as described above and herein, can be administered in combination with one or more additional therapies to treat and/or reduce the symptoms of MS described herein, particularly to treat patients with moderate to severe disability (e.g., EDSS score of 5.5 or higher). The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the pharmaceutical composition with the additional therapeutically active agent and/or the desired therapeutic effect to be achieved. In general, it is expected that additional therapeutic agents utilized in combination be utilized at levels that do not exceed the levels at which they are utilized individually. In some embodiments, the levels utilized in combination will be lower than those utilized individually.

This invention is further illustrated by the following examples which should not be construed as limiting. The contents of all references, figures, sequence listing, patents and published patent applications cited throughout this application are hereby incorporated by reference.

### EXEMPLIFICATION

RRMS is a chronic inflammatory disease which targets the central nervous system. Despite a growing number of approved disease modifying therapies with different mechanisms of action, there is a varied therapeutic response in RRMS patients and an acute need for biomarkers that will identify patients who will respond favorably to therapies either prior to treatment or within a short period on therapy.

Inflammatory proteins including cytokines and chemokines have been shown to be dysregulated in a number of MS subtypes and are linked to pathogenesis. Given the close link between serum proteins and disease state, this study explored the use of disease related protein markers to determine candidate biomarkers of pharmacologic and therapeutic response.

### Example 1

### Sample Population

The serum samples used herein were derived from the subset of 802 subjects enrolled in the intramuscular IFN-β-1A dose comparison study (Biogen C94-805 study). The objective of the study was to compare the efficacy of 30 μg or 60 μg IFN-β-1A delivered intramuscularly once weekly with respect to reducing sustained disability progression. Subjects were enrolled at 38 centers in Europe from 1996 to 1997. All samples from the study were stored at −80° C. This study is described in more detail in Clanet, M. et al. “A randomized, double-blind, dose-comparison study of weekly interferon β-1A in relapsing MS” Neurology (2002) 59:1507-1517, which is herein incorporated by reference in its entirety.

The inclusion criteria for study C94-805 included patients clinically diagnosed with MS for one ore more years, and EDSS score form 2.0 to 5.5, 2 or more relapses in prior 3 years, and stable or improving disease at time of enrollment. The exclusion criteria eliminated individuals with progressive disease (i.e., decline in prior 6 months) and/or those that relapsed within the previous 2 months of enrollment.

TABLE 3
Baseline Patient Demographic and Clinical Characteristics
(Clanet, M et al. Neurology (2002) 59: 1507-1517).

| | IFNβ-1a 30 μg, | IFNβ-1a 60 μg, |
|---|---|---|
| Characteristic | n = 400 | n = 400 |
| Age, y, mean ± SD | 35.9 ± 7.0 | 96.7 ± 7.9 |
| % Women | 66 | 68 |
| % White | 87 | 98 |
| Classification of MS, % | | |
| Relapsing-remitting | 86.0 | 85.5 |
| Relapsing-progressive* | 15.0 | 14.8 |
| Disease duration, y, | 6.6 ± 3.0 | 6.6 ± 6.8 |
| mean ± SD | | |
| Age at diagnosis, y, | 91.3 ± 7.8 | 31.3 ± 7.8 |
| mean ± SD | | |
| EDSS score, mean ± SD | 3.8 ± 1.0 | 3.6 ± 1.0 |
| No. (%) of patients with | | |
| EDSS score: | | |
| ≦2.8 | 335 (68) | 228 (38) |
| 4.0 to 5.5 | 187 (42) | 171 (41) |
| ≧6.9 | 0 (9) | 1 (<1) |
| Prestudy relapse rate,† | 1.3 ± 0.6 | 1.8 ± 0.6 |
| mean ± SD | | |

*Patients with early progressive disease who experienced relapses; patients with confirmed progressive disease and no relapses were excluded from the study.
†Relapse rate per year during the 3 years before study enrollment.
IFN = Interference,
EDSS = Expanded Disability Stains Scale.

402 individuals were assigned to the group receiving the 30 μg AVONEX® (interferon beta 1a) dose, while 400 individuals were assigned to the group receiving the 60 μg AVONEX® (interferon beta 1a) dose. Serum samples were obtained at baseline and at 3 months following initiation of AVONEX® (interferon beta 1a) treatment.

Non-Responders Vs. Responders

Of the combined 802 individuals, 64 were identified as “non-responders (NR)” and 54 individuals were identified as “responders (R).” This subgroup of 118 patients is referred to herein as the “general population of R/NR.”

A “responder” is defined as a subject with no confirmed relapses and no evidence of sustained disability progression (by EDSS) during the first three years of treatment (e.g., clinical remission). A “non-responder” is defined as those subjects that have active disease on therapy including subjects with at least 3 relapses, development of a 6-month sustained progression in disability defined as a 1.0 point increase in EDSS score from baseline in subjects with a baseline score of ≦5.5. Subjects were excluded for having ≧10 MRI T2 lesions in the remission or permanently testing positive for NAB starting from year 1 at any titer or NAB titers ≧20 in either group.

TABLE 4
Subject characteristics for responders and non-responders.

| | Responder | Non-responder |
|---|---|---|
| Characteristic | n = 54 | n = 64 |
| Age, y, mean ± SD | 36.3 ± 9.4 | 37.0 ± 6.9 |
| % Women | 67 | 69 |
| % White | 100 | 98 |
| Classification of MS, % | | |
| Relapsing-remitting | 87 | 85.9 |
| Relapsing-progressive* | 13 | 14.1 |
| Disease Duration, y, mean +/− SD | 4.7 +/− 4.0 | 5.2 +/− 4.4. |
| Age at diagnosis, y, mean ± SD | 32.1 ± 9.1 | 32.3 ± 7.2 |
| EDSS score, mean ± SD | 3.4 ± 1.0 | 3.8 ± 1.1 |
| No. (%) of patients with EDSS score: | | |
| 2.0 to 3.5 | 34 (63.0) | 30 (46.9) |
| 4.0 to 5.5 | 20 (37.0) | 34 (53.1) |
| Prestudy relapse rate**, mean ± SD | 1.0 ± 0.3 | 1.4 ± 0.6 |
| No. (%) of patients on IFNB-1a: | | |
| 30 ug | 25 (46.3) | 32 (50.0) |
| 60 ug | 29 (53.7) | 32 (50.0) |

*Patients with early progressive disease who experienced relapses; patients with confirmed progressive disease and no relapses were excluded from the study.
**Relapse rate per year during the three years before study enrollment.

MRI Subset

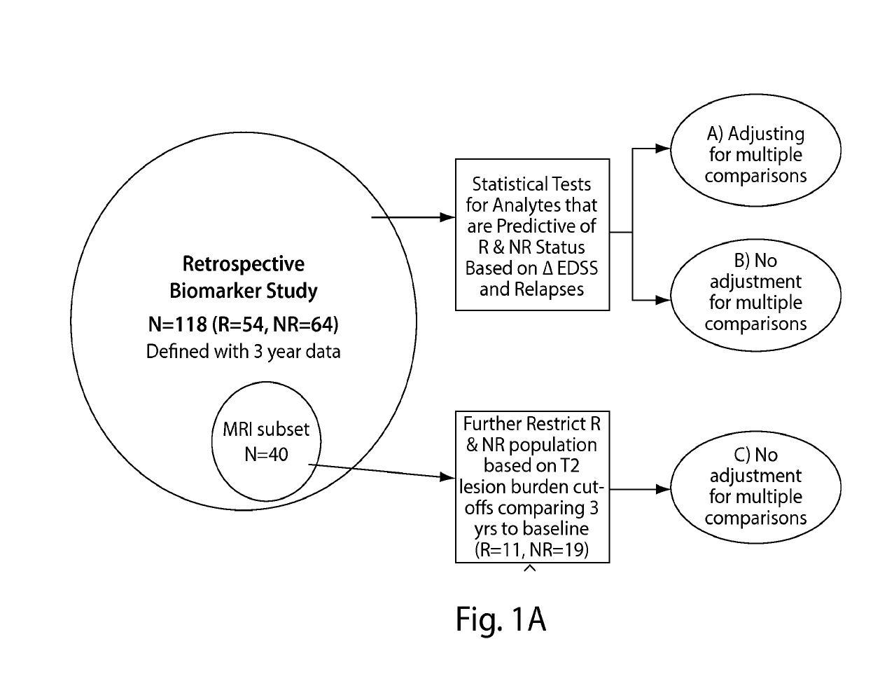

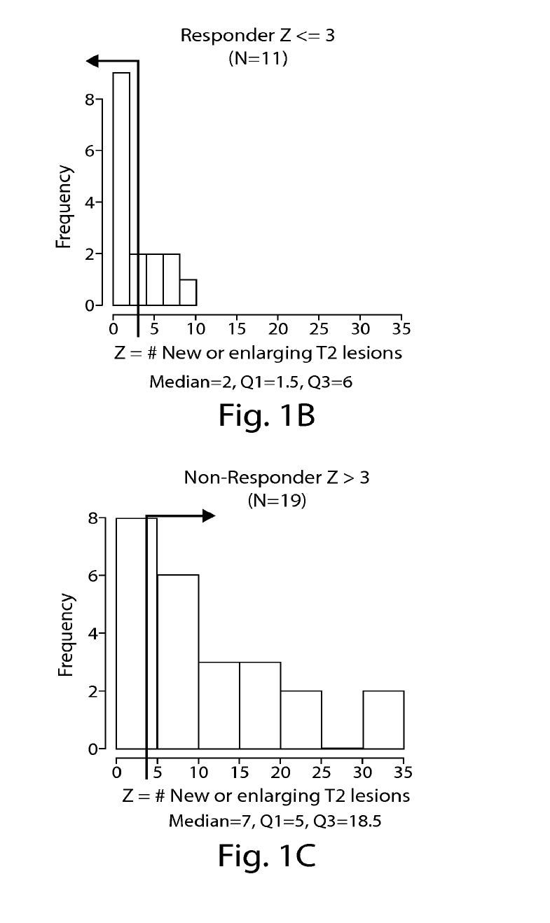

A subset of 40 individuals out of the original sample population of 118 (64 NR and 54 R) underwent MRI to identify the number and size of T2 lesions. Based on the new or enlarging T2 lesions in 3 years, 19 of these individuals were classified as non-responders, while the remaining 11 were classified as responders (FIGS. 1A-1C).

Study Samples

Both pre-treatment and 3-month serum samples were analyzed following ethics committee review. 3-month samples were collected 3 to 7 days following the 3-month dose (12^{th} injection). The protocol called for centrifugation and storage at −20° C. within 1-2 hours of collection. Long-term storage was at −80° C. In addition, fresh serum from healthy volunteers (HV) was collected and stored at −80° C. (BIORECLAMATION INC.).

### Example 2

### Methods and Sample Quality

Analytical Methods

Quantitative measurements of 55 inflammation related proteins were completed for all samples using customized LUMINEX™ assays. The LUMINEX™ assay technology separates tiny color-coded beads into e.g., 500 distinct sets that are each coated with a reagent for a particular bioassay, allowing the capture and detection of specific analytes from a sample in a multiplex manner. The LUMINEX™ assay technology can be compared to a multiplex ELISA assay using bead-based fluorescence cytometry to detect analytes such as biomarkers.

A human inflammation panel was obtained from RULES BASED MEDICINE™ to test for the following inflammation related proteins: IL-17, IL-23, IL-15, IL-7, IL-1α, IL-1β, IL-1RA, IFN-γ, IL-2, IL-3, IL-4, IL-5, IL-6, IL-8, IL-10, IL-12p40, IL-12p70, IL-15, AAT, A2M, B2M, BDNF, CRP, C3, CCL11, F7, FT, FGA, GM-CSF, HB, ICAM-1, MIP-1α, MIP-1β, MMP-2, MMP-3, MMP-9, CCL2, RANTES, SCF, TIMP, TNF-α, TNF-β, TNF-RA2, VCAM-1, VEGF, VWF, and VDBP.

A second panel was custom made for the study and is referred to herein as the Biogen Idec Chemokine Panel. This panel was used to test for the following proteins: CCL19, CCL2, CXCL10, CXCL11, CXCL12, CXCL13, CXCL9, CCL21, and BAFF.

The levels of ferritin and IL-13 were also determined using standard methods.

### Sample Quality

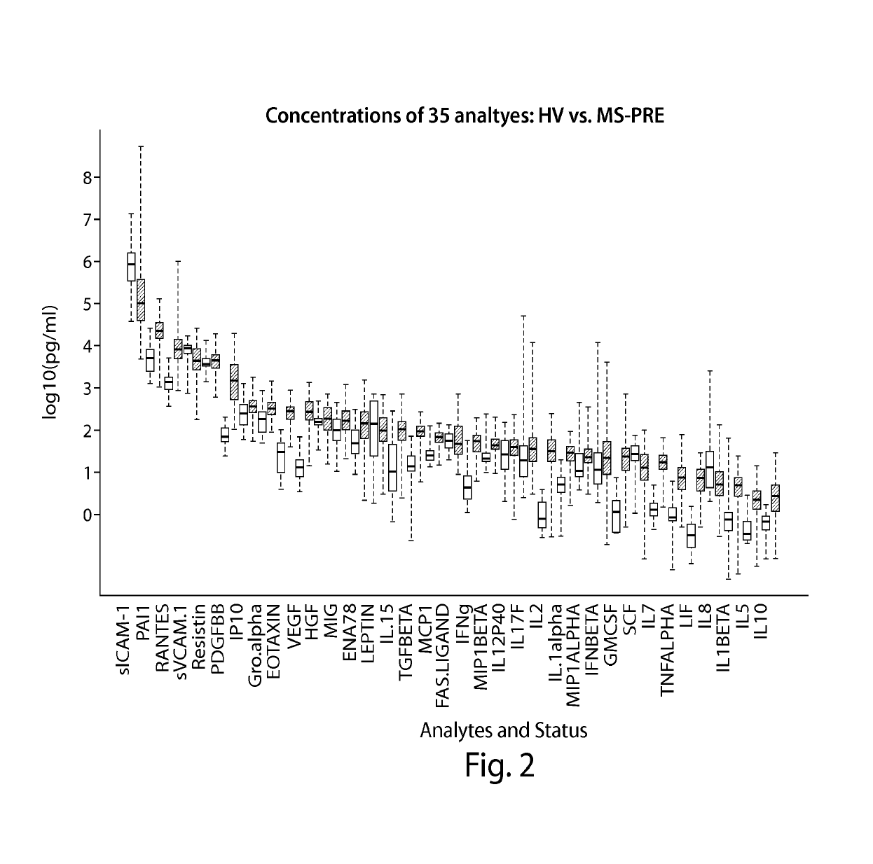

The sample quality of the stored serum samples was compared to fresh serum obtained from healthy volunteers (see FIG. 2). No gross sign of degradation was observed and the concentrations of 35 different analytes were consistent with what was reported in the literature.

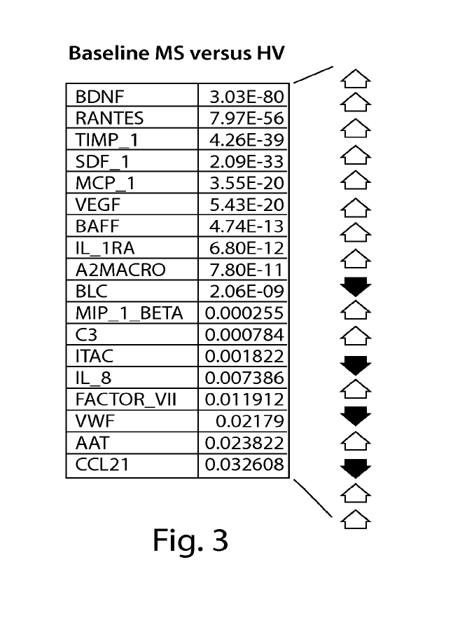

Baseline MS samples have a distinct serum profile compared to those of healthy volunteers, which is consistent with findings in the literature (FIG. 3A). Similar differences were observed after 3-months of treatment with AVONEX® (interferon beta 1a).

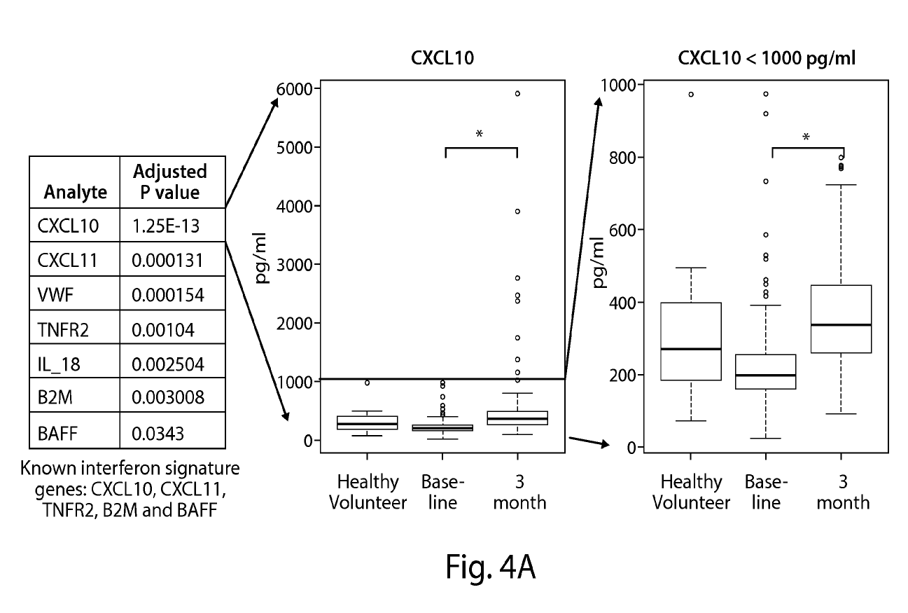

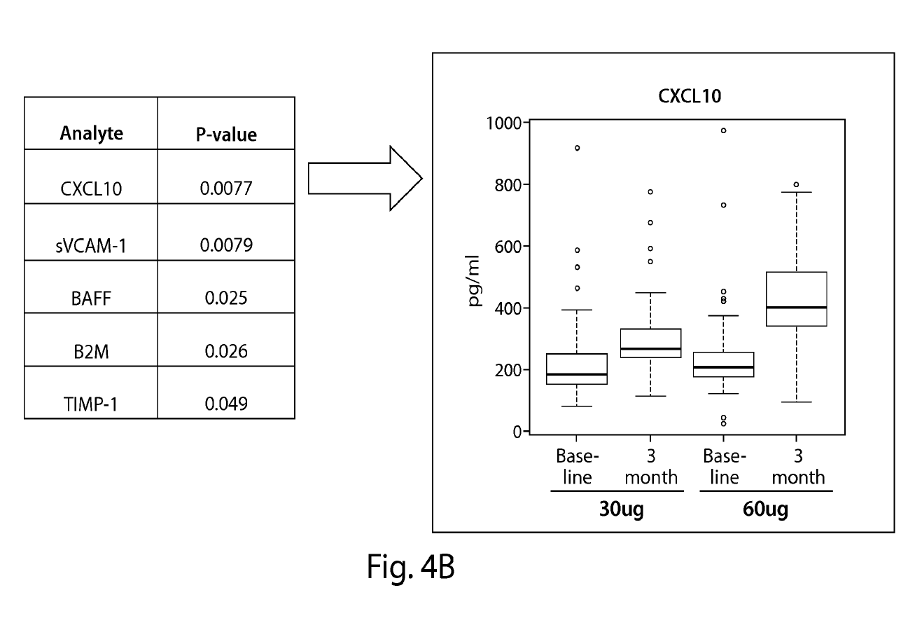

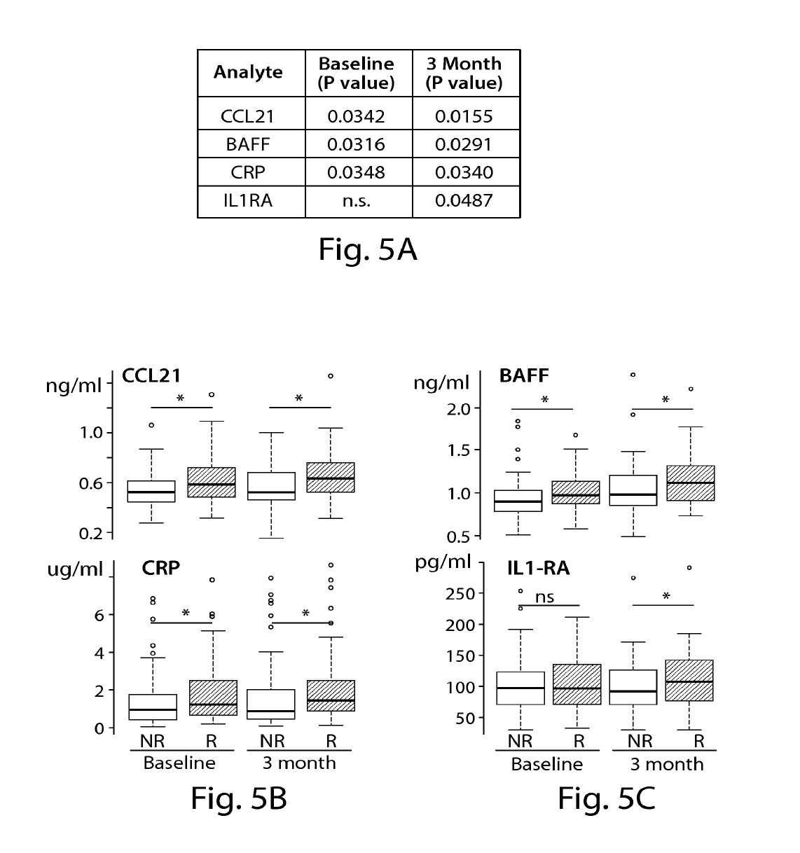

An interferon signature gene response was observed using serum proteins (FIG. 4A) and a dose-dependent response was observed for interferon signature genes between 30 μg and 60 μg doses (FIG. 4B). A comparison of the serum concentrations at baseline versus 3-months is provided in FIGS. 4A-4B). Evidence of a dose dependent pharmacodynamic response after IFNb administration at 30 μg vs. 60 μg is provided in FIG. 4B.

### Example 3

### Predictive Biomarkers of Clinical Response to Intramuscular (IM) IFNβ-1A

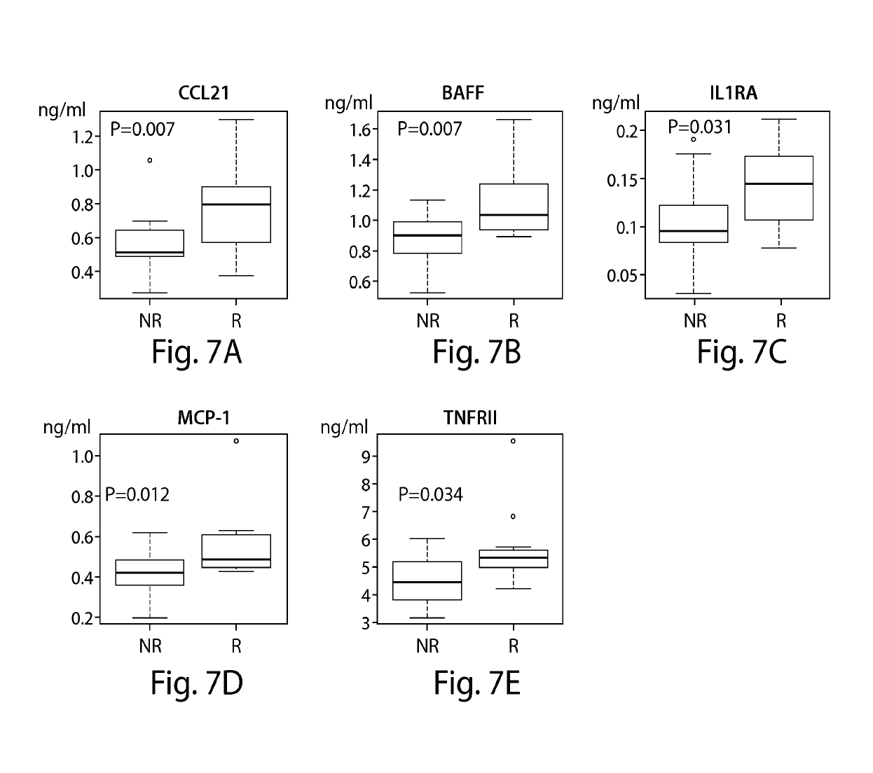

When adjusted for multiple comparisons there were no differences for any analytes from tests using: (i) baseline serum concentration, (ii) 3-month serum concentration, or (iii) concentration difference (ratio of 3-month and baseline). Using raw p-values, expression levels of CCL21, BAFF, CRP, and IL-1RA were determined to be significantly different between responders and non-responders (FIGS. 7A-7E). Thus, CCL21, BAFF, CRP and IL-1RA can be used as biomarkers for classification of those individuals likely to respond to IFNβ-1A treatment and those who will likely remain in an active disease state despite treatment.

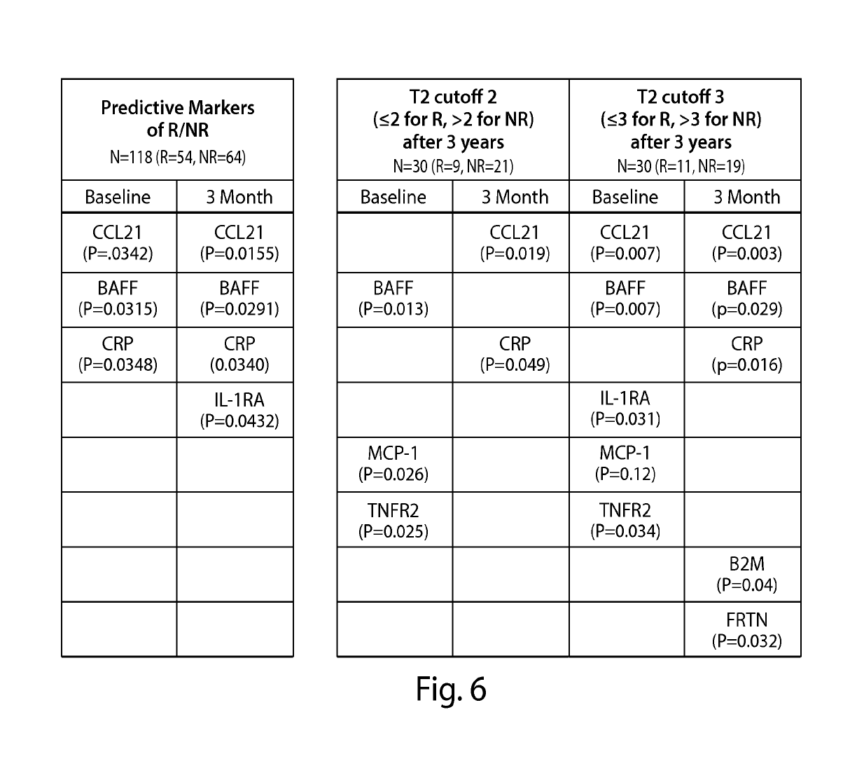

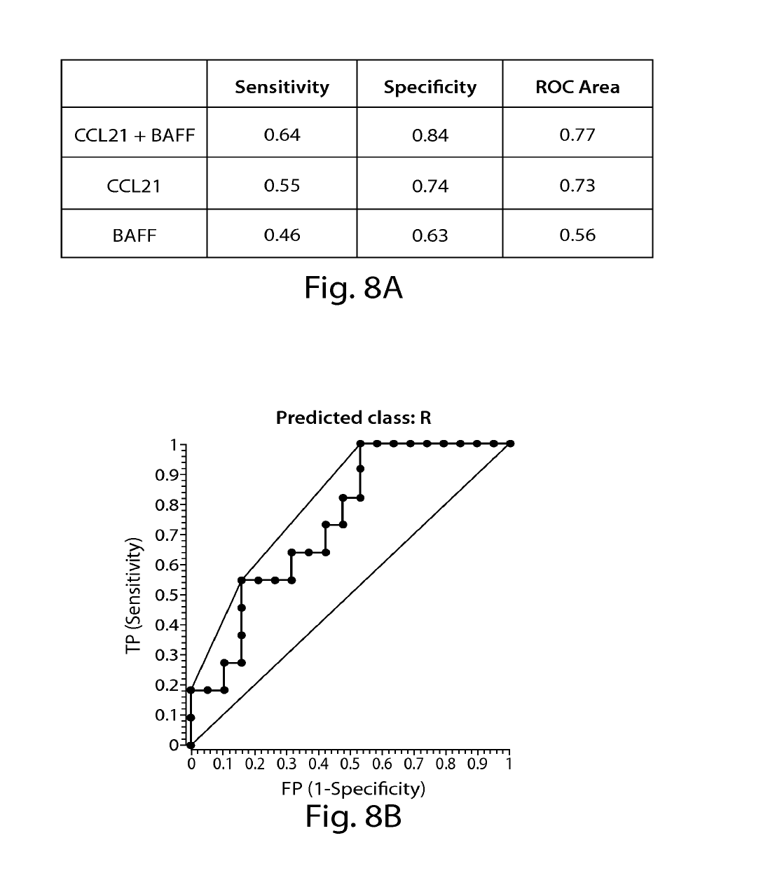

The MRI subset (FIGS. 1A-1C) was also analyzed for predictive markers of therapeutic response. From this subset, the expression of biomarkers CCL21 and BAFF was significantly different (using raw p-values) between non-responders and responders (FIGS. 6-8). Serum levels of CCL21 and BAFF were shown to classify R and NR when using a measure of responder and non-responder which included a combination of EDSS progression, relapse and MRI parameters at 3 years.

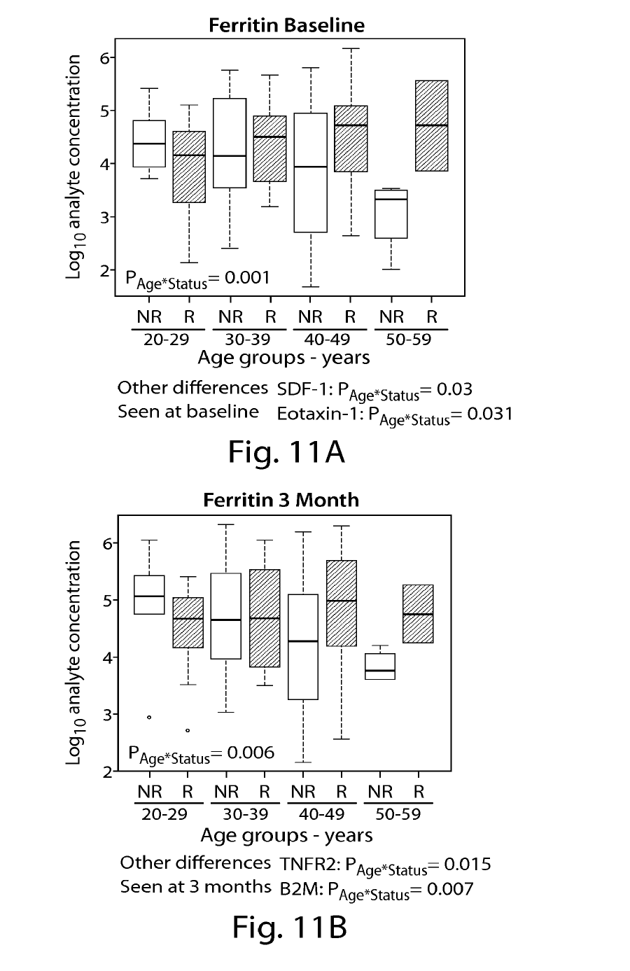

The level of ferritin in each population was also measured. Lower levels of serum ferritin were found to correlate with age and R/NR status at baseline and 3-months of IFNβ-1A therapy using an EDSS and relapse definition (R=54, NR=64; FIGS. 11A-11B).

### Example 4

### Identification of IL-13 as a Biomarker

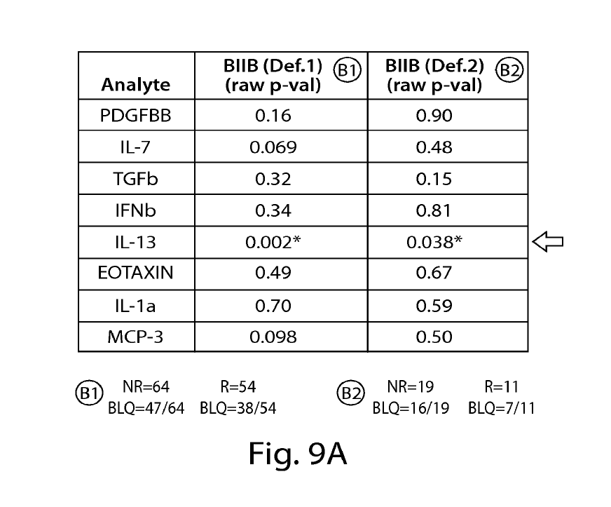

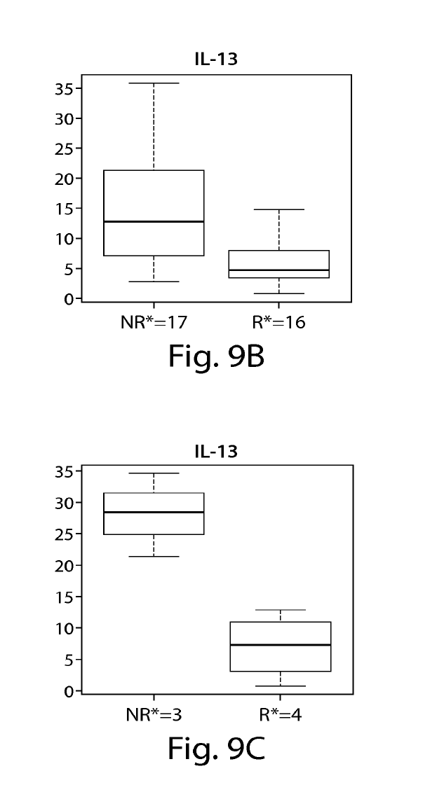

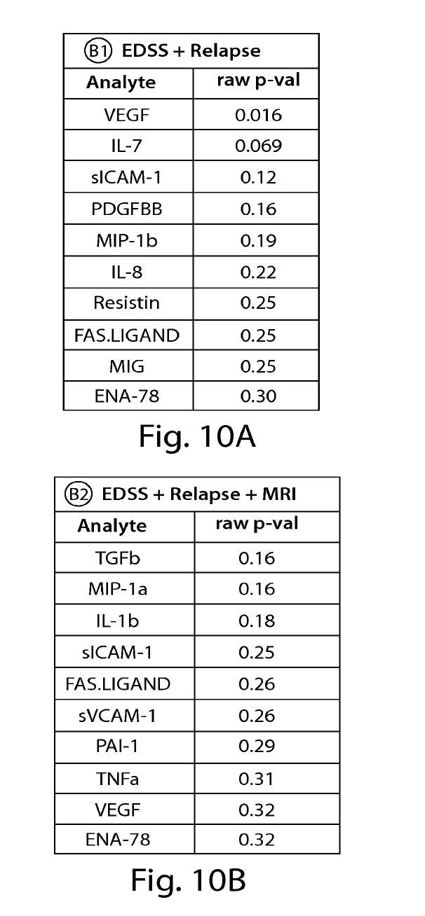

Expression of a set of analytes including PDGFBB, IL-7, TFGb, IFNb, IL-13, Eotaxin, IL-1A and MCP-3 were determined (FIG. 9A; FIGS. 10A-10B) and of this panel only IL-13 was determined to be statistically significant in both the general population of R/NR in this study (B1) and the MRI subset (B2) (FIG. 9A). IL-13 can be used to classify patients as either a non-responder or a responder to IFNβ treatment (FIGS. 9B-9C).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the worldwide web at tigr.org and/or the National Center for Biotechnology Information (NCBI) on the worldwide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed.

What is claimed is:

1. A method of treating or preventing one or more symptoms associated with multiple sclerosis (MS), in a subject having MS, or at risk for developing MS, comprising:
acquiring a value of one or more MS biomarkers chosen from CCL21, BAFF, or a combination thereof, in the subject; and
responsive to said value, administering to the subject an MS treatment that includes an IFN-b agent, in an amount sufficient to reduce one or more symptoms associated with MS,
wherein, in response to an increased value of said MS biomarkers relative to a reference value, the MS treatment is initiated or continued; and
wherein, in response to a decreased value of said MS biomarkers relative to a reference value, the MS treatment is modified.

2. A method for identifying a subject having MS, or at risk for developing MS, as having an increased responsiveness or a decreased responsiveness to an MS treatment that includes an IFN-b agent, comprising:
acquiring a value of one or more MS biomarkers chosen from CCL21, BAFF, or a combination thereof, in the subject; and
responsive to said value, identifying the subject as having the increased or decreased responsiveness to the MS treatment,
wherein, in response to an increased value of said MS biomarkers relative to a reference value, the subject is identified as having the increased responsiveness to the MS treatment; and
wherein, in response to a decreased value in said MS biomarkers relative to a reference value, the subject is identified as having the decreased responsiveness to the MS treatment.

3. A method for evaluating or monitoring a first MS treatment that includes an IFN-b agent in a subject, having MS, or at risk for developing MS, comprising:
acquiring a value of an MS biomarker chosen from CCL21 and BAFF in the subject, prior to, during, and/or after, administering the first MS treatment; and
responsive to said value, administering or altering one or more of:
(i) the first MS treatment, (ii) the dosing of the first MS treatment, (iii) the schedule or time course of the first MS treatment, or (iv) administering a second alternative MS treatment,
wherein, in response to an increased value in said MS biomarkers relative to a reference value, the subject is administered one or more of:
(i) the first MS treatment, (ii) the dosing of the first MS treatment, or (iii) the schedule or time course of the first MS treatment; and
wherein, in response to a decreased value in said MS biomarkers relative to a reference value, the subject is administered a second alternative MS treatment,
thereby evaluating or monitoring the MS treatment.

4. The method of claim 1, wherein a value of CCL21 in the serum of the subject equal to, or higher than, about 0.6 ng/ml is indicative of increased responsiveness of the subject to the MS treatment that includes the IFN-b agent, whereas a CCL21 serum level of less than about 0.6 ng/ml is indicative of decreased responsiveness of the subject to the MS treatment that includes the IFN-b agent.

5. The method of claim 1, wherein a value of BAFF in the serum of the subject equal to, or higher than, about 0.95 ng/ml is indicative of increased responsiveness of the subject to the MS treatment that includes the IFN-b agent, whereas a BAFF serum level of less than about 0.95 ng/ml is indicative of decreased responsiveness to the MS treatment that includes the IFN-b agent.

6. The method of claim 1, wherein the MS biomarkers further comprise one or more of: IL-1RA, IL-13, MCP-1, CRP, B2M, ferritin, or TNFR2.

7. The method of claim 1, wherein the reference value is obtained from one or more of: an MS subject population; or the subject at a different time interval.

8. The method of claim 1, wherein the MS treatment comprises an IFNb agent chosen from an IFN-b 1a molecule, an IFN-b 1b molecule, or a pegylated variant of an IFN-b 1a molecule or an IFN-b 1b molecule.

9. The method of claim 8, wherein the IFNb-1a molecule is Avonex® or Rebif®; and the IFNb-1b molecule is Betaseron® or Betaferon®.

10. The method of claim 1, wherein the MS treatment is modified by administering a second alternative MS treatment.

11. The method of claim 10, wherein the second alternative MS therapy is chosen from:
(i) a polymer of glutamic acid, lysine, alanine and tyrosine or glatiramer;
(ii) an antibody or fragment thereof against alpha-4 integrin or natalizumab;
(iii) an anthracenedione molecule or mitoxantrone;
(iv) a fingolimod or FTY720;
(v) a dimethyl fumarate or an oral dimethyl fumarate
(vi) an antibody to the alpha subunit of the IL-2 receptor of T cells or daclizumab;
(vii) an antibody against CD52 or alemtuzumab; or
(viii) an anti-LINGO-1 antibody.

12. The method of claim 1, wherein the subject is a patient having one of: benign MS, relapsing-remitting multiple sclerosis (RRMS), primary progressive MS, or secondary progressive MS; clinically isolated syndrome (CIS) or clinically defined MS (CDMS).

13. The method of claim 1, wherein the subject is a patient with relapsing-remitting multiple sclerosis (RRMS)).

14. The method of claim 1, wherein the subject is chosen from one or more of: a patient with relapsing-remitting multiple sclerosis (RRMS) prior to administration the MS treatment that includes the IFN-b agent; an RRMS patient during the MS treatment that includes the IFN-b agent; or an RRMS patient after administration of the MS treatment that includes the IFN-b agent.

15. The method of claim 1, wherein said treating or preventing comprises reducing, retarding or preventing, a relapse, or the worsening of a disability, in the MS subject.

16. The method of claim 1, further comprising one or more of: performing a neurological examination, evaluating the subject's status on the Expanded Disability Status Scale (EDSS), or detecting the subject's lesion status as assessed using an MRI.

17. The method of claim 1, further comprising obtaining a sample from the subject, wherein the sample is chosen from a non-cellular body fluid; or a cellular or tissue fraction.

18. The method of claim 17, wherein the non-cellular fraction is chosen from plasma or serum.

19. The method of claim 17, wherein the cellular fraction comprises peripheral blood mononuclear cells (PBMC).

20. The method of claim 16, wherein the subject is monitored in one or more of the following periods: prior to beginning of treatment; during the treatment; or after the MS treatment has been administered.

21. A kit for evaluating a sample from an MS patient, to detect or determine the value of one or more MS biomarkers, comprising a reagent that specifically detects one or more MS biomarkers chosen from CCL21, BAFF, or a combination thereof, with instruction indicating a value of CCL21 or BAFF responsive to an IFN-b therapy.

22. The method of claim 2, further comprising providing or transmitting information or a report, containing data of the evaluation or treatment to a report-receiving party or entity chosen from a patient, a health care provider, a diagnostic provider, or a regulatory agency.

23. A method of, or assay for, evaluating a sample from a subject having multiple sclerosis (MS), or at risk for developing MS, comprising detecting an alteration in at least two MS biomarkers chosen from CCL21 and BAFF in the sample.

24. The method or assay of claim 23, wherein the MS biomarkers further comprise one or more of: IL-1RA, IL-13, MCP-1, CRP, B2M, ferritin or TNFR2.


SEQUENCE LISTING (1 sequence)

SEQ ID NO: 1 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN